(19) United States
(12) Patent Application Publication
(10) Pub. No.: US 2022/0031641 A1
(43) Pub. Date: Feb. 3, 2022

(54) COMPOSITIONS AND METHODS FOR TREATING AND PREVENTING COGNITIVE DYSFUNCTION

(71) Applicants: Pharmatrophix, Inc., Menlo Park, CA (US); The Board and Trustees of the Leland Stanford Junior University, Stanford, CA (US)

(72) Inventors: Michelle Leigh Monje-Deisseroth, Stanford, CA (US); Frank M. Longo, Menlo Park, CA (US)

(21) Appl. No.: 17/382,045

(22) Filed: Jul. 21, 2021

Related U.S. Application Data
(63) Continuation of application No. PCT/US2020/014655, filed on Jan. 22, 2020.
(60) Provisional application No. 62/796,538, filed on Jan. 24, 2019.

(51) Int. Cl. A61K 31/166; A61K 45/06

(52) U.S. Cl. CPC A61K 31/166; A61K 45/06

(57) ABSTRACT

Provided are compositions and methods comprising agonists. Also disclosed are agonist compounds, pharmaceutical compositions of agonists, and methods of using the same.

### CROSS-REFERENCE

This application is a continuation application of International Patent Application PCT/US2020/014655 filed Jan. 22, 2020, which claims the benefit of priority to U.S. Provisional Patent Application No. 62/796,538, filed Jan. 24, 2019, the disclosure of each of which is incorporated herein by reference in its entirety.

### STATEMENT AS TO FEDERALLY SPONSORED RESEARCH

This invention was made with the support of the United States government under Contract number RB4-06093 and RN3-06510 by California Institute for Regenerative Medicine (CIRM).

### BACKGROUND

Despite advances in cancer therapy over the last 50 years, complications associated with conventional cancer therapeutic methods remain, particularly in cases where a cancer therapeutic results in the generation of a secondary disorder.

The disclosed compositions and methods herein can be used for the treatment, amelioration and prevention of cancer therapy-related cognitive dysfunction.

### SUMMARY

In some aspects, the present disclosure provides a method of preventing or treating a cancer therapeutic-related cognitive dysfunction, comprising administering to a subject in need thereof an effective amount of a TrkB agonist, wherein said TrkB agonist is administered to the subject before, during, or after completion of the administration of a cancer treatment regimen or a combination thereof.

In some embodiments, the cancer treatment regimen comprises one or more doses of chemotherapy. In some embodiments, the cancer treatment regimen comprises one or more doses of radiation therapy. In some embodiments, the cancer treatment regimen comprises one or more doses of immunomodulatory therapy.

In some embodiments, the method further comprises administering to the subject a test of cognitive function prior to administration of the TrkB agonist. In some embodiments, the test of cognitive function indicates that the subject has a cognitive dysfunction. In other embodiments, the test of cognitive function indicates that the subject does not have a cognitive dysfunction.

In some embodiments, the cognitive dysfunction comprises one or more symptoms selected from: being unusually disorganized, confusion, difficulty concentrating, difficulty finding the right word, difficulty learning new skills, difficulty multitasking, fatigue, feeling of mental fogginess, short attention span, short-term memory problems, taking longer than usual to complete routine tasks, trouble with verbal memory, adynamia, lack of motivation, impulsiveness, disinhibition, verbosity, tangentiality, irritability, fidgety, low frustration tolerance and trouble with visual memory. In some embodiments, the cognitive dysfunction comprises short-term memory problems.

In some embodiments, the method further comprises administering a test of cognitive function after administration of an effective amount of the TrkB agonist. In some embodiments, the test of cognitive function after administration of an effective amount of the TrkB agonist indicates maintenance or improvement in cognitive function of the subject. In some embodiments, the test of cognitive function after administration of an effective amount of the TrkB agonist indicates improvement in one or more symptoms of cognitive dysfunction of the subject. In some embodiments, the test of cognitive function is selected from the General Practitioner Assessment of Cognition (GPCOG), Memory Impairment Screen (MIS), MiniMental State Exam (MMSE), and the Six Item Cognitive Impairment Test (6CIT).

In some embodiments, the method comprises administering said TrkB agonist to the subject after completion of the administration of a cancer treatment regimen. In some embodiments, the method comprises administering said TrkB agonist to the subject for about 1 month to about 2 years after completion of the administration of the cancer treatment regimen. In some embodiments, the method comprises administering said TrkB agonist to the subject for about 1 month to about 1 year after completion of the administration of the cancer treatment regimen.

In some embodiments, the method comprises administering said TrkB agonist to the subject before the administration of the cancer treatment regimen. In some embodiments, the TrkB agonist is administered to the subject for about 1 day to about 3 months before the administration of the cancer treatment regimen.

In some embodiments, the method comprises administering said TrkB agonist to the subject during the cancer treatment regimen. In some embodiments, the TrkB agonist is administered to the subject within six hours of administration of a dose of the cancer treatment regimen. In some embodiments, the TrkB agonist is administered to the subject within three hours of administration of a dose of the cancer treatment regimen. In some embodiments, the TrkB agonist is administered concurrently with a dose of the cancer treatment regimen.

In some embodiments, the TrkB agonist is administered daily, every other day, every third day, or every fourth day.

In some embodiments, the method comprises promoting or maintaining growth of glial cells. In some embodiments, the glial cells are oligodendrocytes.

In some embodiments, the subject has a cancer selected from breast cancer, ovarian cancer, prostate cancer, leukemia, lymphoma, brain tumor, and sarcoma. In some embodiments, the subject has a cancer selected from a cancer of the central nervous system.

In some embodiments, the subject has a cancer selected from a cancer other than cancer of the central nervous system. In some embodiments, the subject is a human female. In some embodiments, the female subject is pre-menopausal. In some embodiments, the female subject has induced menopause.

In some aspects, the present disclosure provides a method of improving cognition in a healthy subject, comprising administering to said healthy subject an effective amount of a TrkB agonist, wherein healthy subject does not suffer from a cognitive disease or disorder. In some embodiments, the heathy subject has a genetic predisposition to cancer. In some embodiments, the genetic predisposition to cancer is a genetic predisposition to breast cancer, ovarian cancer, colon cancer, rectum cancer, leukemia, lymphoma, brain tumor, or sarcoma.

In some embodiments, the genetic predisposition to cancer comprises a mutation in p53, HER2, BRCA1, BRCA2, or the RAS family. In some embodiments, the genetic predisposition to cancer comprises a family history of cancer. In some embodiments, the healthy subject does not have a disease selected from the following: Alzheimer's disease, Huntington's disease, amyotrophic lateral sclerosis, Rett syndrome, epilepsy, Parkinson's disease, spinal cord injury, stroke, hypoxia, ischemia, brain injury, diabetic neuropathy, peripheral neuropathy, nerve transplantation complications, motor neuron disease, multiple sclerosis, HIV dementia, peripheral nerve injury, hearing loss, depression, obesity, metabolic syndrome, pain, and cancer.

In some embodiments, the TrkB agonist is a BDNF mimetic. In some embodiments, the TrkB agonist is represented by Formula (II):

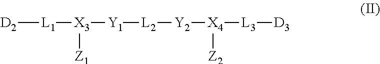

wherein:

L₁ and L₃ are independently selected from the group consisting of C₁-C₅ alkylene, arylene, aralkylene, and substituted arylene;

L₂ is selected from the group consisting of C₁-C₅ alkylene, arylene, aralkylene, substituted arylene,

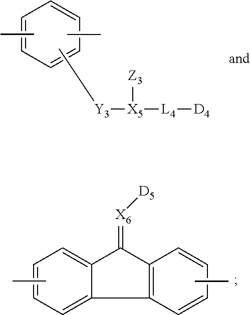

L₄ is C₁-C₅ alkylene;

Z₁, Z₂, and Z₃ are independently selected from the group consisting of H, alkyl, aryl, and aralkyl;
X₃, X₄, X₅, and X₆ are independently N or CH;

Y₁, Y₂, and Y₃ are independently carbonyl, sulfonyl, or methylene; and

D₂, D₃, D₄, and D₅ are independently selected from H, alkyl, halo, hydroxyl, mercapto, mercaptoalkyl, alkoxyl, aryloxyl, aralkoxyl, acyloxyl, carboxyl, alkyloxycarbonyl, aryloxycarbonyl, aralkoxycarbonyl, acylamino, carbamoyl, alkylcarbamoyl, dialkylcarbamoyl,

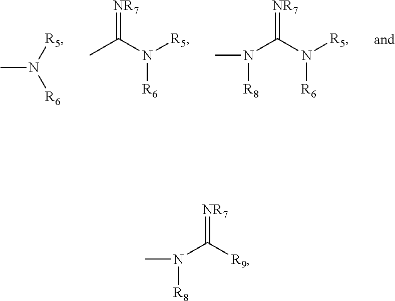

wherein R₅, R₆, R₇, R₈, and R₉ are independently selected from H, alkyl, aralkyl, and aryl;

or a pharmaceutically acceptable salt thereof.

In some embodiments, the compound is selected from the group consisting of:

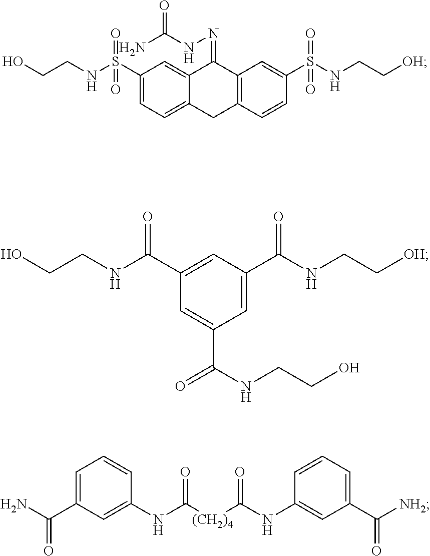

and a pharmaceutically acceptable salt of any one thereof.

In some embodiments, the method further comprises administering a second therapeutic agent selected from an agent that promotes myelination. In some embodiments, the second therapeutic agent is selected from a parasympathomimetic drug, a Glycogensynthase kinase 3 beta inhibitor and a sterol.

In some embodiments, the present disclosure provides a pharmaceutical formulation, comprising:

(a) a compound of Formula (II):

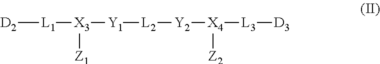

wherein:

L₁ and L₃ are independently selected from the group consisting of C₁-C₅ alkylene, arylene, aralkylene, and substituted arylene;

L₂ is selected from the group consisting of C₁-C₅ alkylene, arylene, aralkylene, substituted arylene,

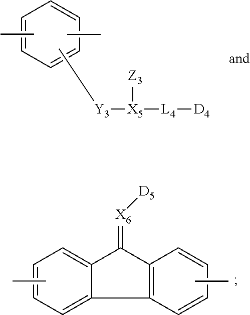

L₄ is C₁-C₅ alkylene;

Z₁, Z₂, and Z₃ are independently selected from the group consisting of H, alkyl, aryl, and aralkyl;
X₃, X₄, X₅, and X₆ are independently N or CH;

Y₁, Y₂, and Y₃ are independently carbonyl, sulfonyl, or methylene; and

D₂, D₃, D₄, and D₅ are independently selected from H, alkyl, halo, hydroxyl, mercapto, mercaptoalkyl, alkoxyl, aryloxyl, aralkoxyl, acyloxyl, carboxyl, alkyloxycarbonyl, aryloxycarbonyl, aralkoxycarbonyl, acylamino, carbamoyl, alkylcarbamoyl, dialkylcarbamoyl,

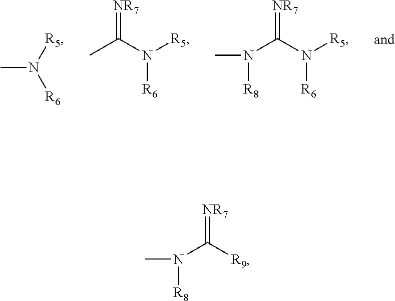

wherein R₅, R₆, R₇, R₅, and R₉ are independently selected from H, alkyl, aralkyl, and aryl;

or a pharmaceutically acceptable salt thereof;
(b) a second therapeutic agent; and
(c) a pharmaceutically acceptable excipient.

In some embodiments, the second therapeutic agent is selected from a parasympathomimetic drug, a Glycogensynthase kinase 3 beta inhibitor, and a sterol.

### INCORPORATION BY REFERENCE

All publications, patents, and patent applications herein are incorporated by reference in their entirety to the same extent as if each individual publication, patent, or patent application was specifically and individually indicated to be incorporated by reference. In the event of a conflict between a term herein and a term in an incorporated reference, the term herein controls.

### BRIEF DESCRIPTION OF THE DRAWINGS

The novel features of the disclosure are set forth with particularity in the appended claims. A better understanding of the features and advantages of the disclosure will be obtained by reference to the following detailed description that sets forth illustrative embodiments, in which the principles of the disclosure can be utilized, and the accompanying drawings of which:

FIG. 1A shows a schematic of a BdnfTMKI genetic mouse model, which lacks activity-regulated BDNF expression. FIG. 1B shows verification of successful knockdown of TrkB in OPCs following recombination induced by tamoxifen administration. Immunohistological analysis of the percentage of PDGFRa+ OPCS co-expressing TrkB (n=4 mice each group). FIG. 1C shows a schematic of optogenetic ferrule placement in superficial cortex of the premotor (M2) region of the mouse brain and blue light stimulation of Thy1::ChR2+/− layer V/VI projection neurons (red). FIG. 1D shows a schematic of single optogenetic stimulation paradigm in BdnfTMKI; Thy1::ChR2+/− mice and Pdgfra-CreERT2; TrkBfl/fl; Thy1::ChR2+/− (OPC-TrkB cKO) mice. Tamoxifen was injected for five consecutive days (100 mg/kg, P24-28, TrkB cKO model only). Optogenetic ferrules were placed on P28, and on P35 a single optogenetic stimulation session (30 sec on/120 sec off, 473 nm light at 20 Hz for 30 minutes) was administered and animals sacrificed three hours following completion of the session. FIG. 1E shows density of proliferating OPCs (EdU+/PDGFRa+) cells in the premotor projections of the corpus callosum of BdnfTMKI; Thy1::ChR2+/− mice (n=10), BdnfTMKI; WT (no opsin) mice (n=8), BdnfWT; Thy1::ChR2+/− (n=7 mice) and BdnfWT; WT (no opsin) mice (n=9) at 3 hours following a single optogenetic stimulation session. FIG. 1F shows representative confocal images of PDGFRa+ OPCs (green) colocalized with EdU+ cells (red) in the corpus callosum (scale bars=20 μm). FIG. 1G shows total density of proliferating OPCs (EdU+/PDGFRa+ cells) in the corpus callosum of OPC-TrkB cKO; Thy1::ChR2+/− mice (n=8), OPC-TrkB cKO; WT mice (n=10) and identically manipulated TrkB WT; Thy1::ChR2+/− mice (n=8) and TrkB WT; WT mice (n=7) 3 hours following a single optogenetic stimulation session. FIG. 1H shows representative confocal images of PDGFRa+ OPCs (green) co-localized with EdU+ cells (red) in the corpus callosum. Scale bars=20 μm. FIG. 1I shows total BDNF protein levels in brain tissue are decreased in the BdnfTMKI model compared to BdnfWT control mice (n=4 mice in each group, p=0.003). Frontal cortex and corpus callosum white matter tissue microdissected at P21 and protein levels examined via ELISA. Data shown in FIG. 1A-FIG. 1I is depicted as mean±SEM, ns=p>0.05, ** p<0.01. Student's t-test (B); Two-way ANOVA with Tukey post-hoc analysis for multiple comparisons (FIG. 1E and FIG. 1G).

FIG. 2A shows a schematic of one-week optogenetic stimulation paradigm in BdnfTMKI; Thy1::ChR2+/− mice and Pdgfra-CreERT2; TrkBfl/fl; Thy1::ChR2+/−(OPC-TrkB cKO) mice. Tamoxifen was injected for five consecutive days (100 mg/kg, P24-28, TrkB cKO model only). Optogenetic ferrules were placed on P28, and beginning on P35 mice received daily optogenetic stimulation sessions (10 min/day, 30 sec on/120 sec off, 473 nm light at 20 Hz) for one week. Mice were sacrificed for histological analyses four weeks later (P69). FIG. 2B shows density of EdU-marked, oligodendrocytes (EdU+/CC1+ cells) in the premotor projections of the corpus callosum of BdnfTMKI; Thy1::ChR2+/− mice (n=6), BdnfTMKI; WT mice (n=5), BdnfWT; Thy1::ChR2+/− mice (n=5) and BdnfWT; WT mice (n=6) 4 weeks after completion of the optogenetic stimulation paradigm. FIG. 2C shows representative confocal images of oligodendrocytes (CC1+, green) co-localized with EdU (red) in the premotor projections of the corpus callosum. Scale bars=20 μm. FIG. 2D shows total density of EdU-marked oligodendrocytes (EdU+/CC1+ cells) in the premotor projections of the corpus callosum of OPC TrkB cKO; Thy1::ChR2+/− mice (n=6), OPC TrkB cKO; WT (no opsin) mice (n=6), TrkB WT; Thy1::ChR2+/− mice (n=6) and TrkB WT; WT (no opsin) mice (n=6) 4 weeks after completion of the optogenetic stimulation paradigm. FIG. 2E shows representative confocal images of oligodendrocytes (CC1+, green) co-localized with EdU (red) in the premotor projections of the corpus callosum. Scale bar=20 μm. Data shown in FIG. 2A-FIG. 2E shown as mean±SEM, ns=p>0.05, **** p<0.0001. Two-way ANOVA with Tukey post-hoc analysis for multiple comparisons (FIG. 2B and FIG. 2D).

FIG. 3A shows a schematic of one-week optogenetic stimulation paradigm as in FIG. 2A. FIG. 3B shows transmission electron microscopy (EM) performed 4-weeks following the cessation of the one-week optogenetic stimulation paradigm. Myelin sheath thickness was analyzed at the level of the cingulum of the corpus callosum as the g-ratio (diameter of axon divided by the diameter of axon plus myelin sheath; a smaller g-ratio indicates a thicker myelin sheath). Scatterplot of g-ratios as a function of axon caliber shows an increase in myelin sheath thickness (smaller g-ratio) in BdnfWT; Thy1::ChR2+/− mice (n=6; red triangles) compared to BdnfTMKI; Thy1::ChR2+/− mice (n=6; black triangles). A single point indicates the g-ratio for a single axon. Approximately 100 axons were quantified and the mean g-ratio determined for each mouse. P-values (indicated on plots) were determined by a Student's t-test, comparing the mean g-ratio on a per mouse basis between groups. FIG. 3C shows bar graphs representing the g-ratio data shown above in B, expressed as the mean g-ratio±SEM for each group of mice in the BdnfTMKI; Thy1::ChR2+/−model (n=6 mice in each group).

FIG. 3D shows representative EM images of premotor projections in cross section at the level of the cingulum of the corpus callosum, as in C. Scale bars=2 μm. FIG. 3E shows transmission electron microscopy (EM) was performed 4-weeks following the cessation of the one-week optogenetic stimulation paradigm. Myelin sheath thickness was analyzed at the level of the cingulum of the corpus callosum as the g-ratio (diameter of axon divided by the diameter of axon plus myelin sheath; a smaller g-ratio indicates a thicker myelin sheath). Scatterplot of g-ratios as a function of axon caliber shows an increase in myelin sheath thickness (smaller g-ratio) in TrkB WT; Thy1::ChR2+/mice− (n=7; red triangles) compared to OPC-TrkB cKO; Thy1::ChR2+/− mice (n=7; black triangles). A single point indicates the g-ratio for a single axon. Approximately 100 axons were quantified and the mean g-ratio determined for each mouse. P-values (indicated on plots) were determined by a Student's t-test, comparing the mean g-ratio on a per mouse basis between groups. FIG. 3F shows bar graphs representing the g-ratio data shown above in E, expressed as the mean g-ratio±SEM for each group of mice in the OPC-TrkB cKO; Thy1::ChR2+/− model (n=7 mice in each group). FIG. 3G shows representative TEM images of premotor projections in cross section at the level of the cingulum of the corpus callosum, as in F. Scale bars=2 μm. FIG. 3H to FIG. 3K show transmission electron microscopy (EM) was performed 4-weeks following the cessation of the one-week optogenetic stimulation paradigm. Myelin sheath thickness was analyzed at the level of the cingulum of the corpus callosum as the g-ratio (diameter of axon divided by the diameter of axon plus myelin sheath; a smaller g-ratio indicates a thicker myelin sheath). Scatterplots of g-ratio as a function of axon caliber are shown comparing optogenetically stimulated mice vs. identically manipulated WT controls for each genotype studied. A single point indicates the g-ratio for a single axon. Approximately 100 axons were quantified and the mean g-ratio determined for each mouse. P-values (indicated on plots) were determined by a Student's t-test, comparing the mean g-ratio on a per mouse basis between groups. FIG. 3H shows g-ratios shown as a scatterplot of all axons measured in BdnfWT; Thy1::ChR2+/− (n=6 mice; red triangles) mice compared to BdnfWT; WT (n=6 mice; grey squares). FIG. 3I shows g-ratios shown as a scatterplot of all axons measured in BdnfTMKI; WT (n=6 mice; blue circles) vs. BdnfTMKI; Thy1::ChR2+/− (n=6 mice; black triangles) show no difference in myelin sheath thickness. FIG. 3J shows g-ratios shown as a scatterplot of all axons measured in TrkB WT; WT (n=7 mice; grey squares) vs. TrkB WT; Thy1::ChR2+/− (n=7 mice; red triangles). FIG. 3K shows g-ratios shown as a scatterplot of all axons measured in OPC-TrkB cKO; WT (n=7 mice; blue circles) vs. OPC-TrkB cKO; Thy1::ChR2+/− (n=7 mice; black triangles) show no difference in myelin sheath thickness. Data shown in FIG. 3A-FIG. 3G shown as mean±SEM, scale bars are 2 μm. ns=p>0.05, * p<0.05. Student's t-tests were run for FIG. 3B, FIG. 3C, FIG. 3E, and FIG. 3F.

FIG. 4A shows a schematic of methotrexate chemotherapy treatment (100 mg/kg methotrexate (MTX) or PBS vehicle control administered i.p. at P21, 28 and 35) in Thy1::ChR2+/− or WT (no opsin) mice followed by a single optogenetic stimulation session of M2 premotor cortex as in FIG. 1A. Mice were sacrificed for analysis 3 hours following the completion of the optogenetic stimulation session. FIG. 4B shows density of EdU-marked OPCs in the corpus callosum of Thy1::ChR2+/− mice (n=4) and identically manipulated WT (no opsin) mice (n=4) that were previously exposed to MTX or PBS vehicle control at 3-hours following a single optogenetic stimulation session. FIG. 4C shows a schematic of juvenile chemotherapy treatment paradigm from P21-P35, followed one month later by the one-week optogenetic stimulation paradigm as in FIG. 2A. Mice were sacrificed at P98, one month following the completion of the week-long optogenetic stimulation paradigm. FIG. 4D and FIG. 4E show Transmission electron microscopy (EM) was performed one month following the cessation of the optogenetic stimulation paradigm in Thy1::ChR2+/− mice that were either stimulated or identically manipulated with the exception of blue light exposure (unstimulated controls). These Thy1::ChR2+/− mice were previously exposed to MTX or PBS vehicle control, resulting in four experimental groups. The g-ratio data are shown as a function of axon caliber as a scatterplot of all axons measured in FIG. 4D PBS vehicle control-treated, unstimulated mice (n=6; black triangles) compared to PBS vehicle control-treated, optogenetically stimulated mice (n=5; red triangles) and in FIG. 4E MTX-treated, unstimulated mice (n=4; grey triangles) compared to MTX-treated, optogenetically stimulated mice (n=3; blue triangles). A single point indicates the g-ratio for a single axon. Approximately 100 axons were quantified and the mean g-ratio determined for each mouse. P-values (indicated on plots) were determined by a Student's t-test, comparing the mean g-ratio on a per mouse basis between groups. FIG. 4F shows representative EM images of M2 projections entering the subjacent corpus callosum. Scale bars=2 μm. FIG. 4G shows total BDNF protein levels from corpus callosum tissue microdissected at P63 and measured by ELISA in mice treated with MTX (n=6 mice) or PBS vehicle control (n=5 mice). Data shown as mean±SEM, ns=p>0.05, * p<0.05, ** p<0.01. Student's t-test run on FIG. 4B, FIG. 4D, FIG. 4E, and FIG. 4G.

FIG. 5A shows a six-point dose response curve demonstrating OPC proliferation index as measured by EdU+/PDGFRa+co-labeling of P6-8 mouse OPCs 24 hours after exposure to recombinant BDNF at a 0-1000 nM concentration range (n=4 wells per condition). FIG. 5B shows a four-point dose response curve demonstrating OPC proliferation index as measured by EdU+/PDGFRa+co-labeling of P6-8 mouse OPCs 24 hours after exposure to LM22A-4 at a 0-1000 nM concentration range (n=4 wells per condition). FIG. 5C shows percent change in CC1+ cells relative to vehicle control increases 7 days after daily treatment with BDNF (50 nM) or LM22A-4 (100 nM) of P6-8 mouse OPCs (n=6 wells per condition). FIG. 5D shows human iPS cells were treated with one of three treatment paradigms from day 25-35 in the OPC induction protocol: no BDNF, 20 ng/ml BDNF as per standard protocol, or BDNF was replaced with 1000 nM LM22A-4. Cells were fixed at day 100 following induction and the percent of DAPI+ cells expressing the OPC marker PDGFRa was quantified. FIG. 5E shows a representative image of a human iPSC-derived PDGFRa+ OPC. Green=PDGFRa, white=DAPI. Scale bar=10 μm. FIG. 5F shows results from a study where human iPS cells were treated with one of three treatment paradigms from day 25-35 in the OPC induction protocol: no BDNF, 20 ng/ml BDNF as per standard protocol, or BDNF was replaced with 1000 nM LM22A-4. Cells were fixed at day 100 following induction and the percent of DAPI+ cells expressing the mature oligodendrocyte marker MBP was quantified. FIG. 5G shows a representative image of a human iPSC-derived MBP+ oligodendrocyte. Red=MBP, white=DAPI. Scale bar=10 μm. FIG. 5H shows a representative image of lysolecithin-induced demyelination in the corpus callosum 10 days after injection. Green=fluoromyelin stain to demonstrate myelinated architecture. White-dashed circle=lesioned area in the corpus callosum. Scale bar=20 μm. FIG. 5I shows total density of EdU+/PDGFRa+ proliferating OPCs in the corpus callosum of mice treated with 50 mg/kg LM22A-4 or vehicle control for 7 consecutive days beginning 7 days after lysolecithin injection (n=8 mice in each group). FIG. 5J shows total density of EdU-marked oligodendrocytes (EdU+/CC1+) in the corpus callosum of mice treated with 50 mg/kg LM22A-4 or vehicle control for 14 consecutive days beginning 7 days after lysolecithin injection (n=7 mice in each group). FIG. 5K-FIG. 5M show representative confocal images of proliferating OPCs as shown by EdU incorporation into PDGFRa+ cells in mouse OPC cultures treated with vehicle control (FIG. 5K), 50 nM BDNF (FIG. 5L) and 100 nM LM22A-4 (FIG. 5M). Scale bar=50 μm. shows Data from FIG. 5A-FIG. 5J shown as mean±SEM. ns=p>0.05, * p<0.05, ** p<0.01, *** p<0.001, **** p<0.0001 as determined by Student's t-test.

FIG. 6A shows a schematic of methotrexate chemotherapy treatment (100 mg/kg methotrexate (MTX) or PBS administered i.p. at P21, 28 and 35) in mice followed by daily injections of 50 mg/kg LM22A-4 or vehicle control from P38-P63. FIG. 6B shows transmission electron microscopy (EM) was performed at P63. g-ratios (diameter of axon/diameter of axon plus myelin sheath) were measured in the projections from M2 at the level of the cingulum of the corpus callosum. Scatterplot of g-ratios as a function of axon caliber demonstrates an increase in myelin sheath thickness (smaller g-ratio) in MTX+LM22A-4-treated mice (n=7 mice; red triangles) compared to MTX+vehicle control-treated mice (n=7 mice; black triangles). A single point indicates the g-ratio for a single axon. Approximately 100 axons were quantified and the mean g-ratio determined for each mouse. P-values (indicated on plots) were determined by a Student's t-test, comparing the mean g-ratio on a per mouse basis between groups. FIG. 6C shows bar graphs representing the g-ratio data shown above in B, expressed as the mean g-ratio±SEM for each group of mice measured (n=7 mice in each group). FIG. 6D shows Novel Object Recognition Test of attention and memory function. At P63, the Novel Object Recognition Test was performed in mice previously exposed to MTX chemotherapy or PBS vehicle control and later treated with LM22A-4 or vehicle control. Percent preference for novel object was measured as the percent time spent with novel object over the total time spent with either object (n=4 mice in each group). Statistical significance was determined by a one-sample t-test, where the average percent preference for the novel object for each group was measured against a hypothetical value of 50% chance an animal would interact with either object. LM22A-4 rescues the impairment that MTX-treated mice exhibit in this behavioral test. FIG. 6E shows representative EM images of M2 projections entering the subjacent corpus callosum. Scale bars=2 μm. FIG. 6F-FIG. 6G show myelin microstructure data shown in FIG. 6C are shown as scatterplots of g-ratio as a function of axon caliber. FIG. 6F shows g-ratio values as a function of axon caliber are shown as a scatterplot of all axons measured in mice treated with PBS vehicle control+vehicle control (n=7 mice; blue circle) vs PBS vehicle control +LM22A-4 (n=7 mice; grey square). LM22A-4 increases myelin sheath thickness in control mice. FIG. 6G shows g-ratio values as a function of axon caliber are shown as a scatterplot of all axons measured in mice treated with PBS vehicle control+vehicle control (n=7 mice; blue circle) vs. MTX+vehicle control (n=7 mice; black triangle). MTX-treated mice exhibit thinner myelin compared to PBS vehicle-treated controls. FIG. 6H shows myelin microstructure data shown in FIG. 6A-FIG. 6E are broken down by axon size. LM22A-4 rescues myelin deficits in mice exposed to juvenile MTX in small caliber axons (<0.5 μm; n=7 mice, p=0.005), medium caliber axons (0.5 μm-1.0 μm; n=7, p=0.006) and large caliber axons (>1.0 μm; n=7 mice; p=0.033) compared to mice exposed to MTX but treated with vehicle control. Data in FIG. 6A-FIG. 6E shown as mean±SEM, ns=p>0.05, * p<0.05, ** p<0.01 *** p<0.001 as determined by Student's t-test.

### DETAILED DESCRIPTION

The following description and examples illustrate embodiments of the disclosure in detail. It is to be understood that this disclosure is not limited to the particular embodiments described herein and as such can vary. Those of skill in the art will recognize that there are numerous variations and modifications of the disclosure, which are encompassed within its scope.

### Definitions

Unless defined otherwise, all technical and scientific terms used herein have the same meaning as is commonly understood by one of skill in the art to which this invention belongs.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

The terms “administer”, “administered”, “administers” and “administering” are defined as the providing a compound or composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. Administration can be continuous or intermittent. In various aspects, a compound or composition disclosed herein can be administered therapeutically. In some instances a compound or composition can be administered to treat an existing disease or condition. In further various aspects, a compound or composition can be administered prophylactically to prevent a disease or condition.

The term “agonist” as used herein can refer a “full agonist” or a “partial agonist.” A partial agonist does not elicit the maximum possible response that is produced by full agonists. The maximum response produced by a partial agonist is called its intrinsic activity and may be expressed on a percentage scale where a full agonist produces a 100% response. A partial agonist may display both agonistic and antagonistic effects. For example, in the presence of a full agonist, a partial agonist may act as an antagonist, competing with the full agonist for the same receptor and thereby reducing the ability of the full agonist to produce its maximum effect. The balance of activity between agonist and antagonist effects varies from one substance to another, according to their intrinsic activities and is also influenced by the test system used to measure the effects.

The term “cancer” and its grammatical equivalents as used herein can refer to a hyperproliferation of cells whose unique trait-loss of normal controls-results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, rectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, sarcoma, skin cancer, small intestine cancer, soft tissue cancer, solid tumors, stomach cancer, testicular cancer, thyroid cancer, ureter cancer, and/or urinary bladder cancer. As used herein, the term “tumor” refers to an abnormal growth of cells or tissues, e.g., of malignant type or benign type.

A “chemotherapeutic agent” or “chemotherapeutic compound” and their grammatical equivalents as used herein, can be a chemical compound useful in the treatment of a disease, for example cancer.

The term “function” and its grammatical equivalents as used herein can refer to the capability of operating, having, or serving an intended purpose. Functional can comprise any percent from baseline to 100% of an intended purpose. For example, functional can comprise or comprise about 5, 10, 15, 20, 25, 30, 35, 40, 45, 50, 55, 60, 65, 70, 75, 80, 85, 90, 95, or up to about 100% of an intended purpose. In some cases, the term functional can mean over or over about 100% of normal function, for example, 125, 150, 175, 200, 250, 300%, 400%, 500%, 600%, 700% or up to about 1000% of an intended purpose.

The term “pharmaceutically acceptable carrier” and their grammatical equivalents can refer to sterile aqueous or non-aqueous solutions, dispersions, suspensions or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These solutions, dispersions, suspensions or emulsions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly (orthoesters) and poly (anhydrides).

The term “subject” and its grammatical equivalents as used herein can refer to a human or a non-human. A subject can be a mammal. A subject can be a human mammal of a male or female gender. A subject can be of any age. A subject can be an embryo. A subject can be a newborn or up to about 100 years of age. A subject can be in need thereof. A subject can have a disease such as cancer. A subject can be premenopausal, menopausal, or have induced menopause. In some embodiments, the subject can be a heathy subject. A healthy subject may not suffer from a cognitive disease or disorder. The healthy subject may have a genetic predisposition to cancer.

The terms “treatment” or “treating” and their grammatical equivalents can refer to the medical management of a subject with the intent to cure, ameliorate, stabilize, or prevent a disease, condition, or disorder. Treatment can include active treatment, that is, treatment directed specifically toward the improvement of a disease, condition, or disorder. Treatment can include causal treatment, that is, treatment directed toward removal of the cause of the associated disease, condition, or disorder. In addition, this treatment can include palliative treatment, that is, treatment designed for the relief of symptoms rather than the curing of the disease, condition, or disorder. Treatment can include preventative treatment, that is, treatment directed to minimizing or partially or completely inhibiting the development of a disease, condition, or disorder. Treatment can include supportive treatment, that is, treatment employed to supplement another specific therapy directed toward the improvement of the disease, condition, or disorder. In some instances, a condition can be pathological. In some instances, a treatment may not completely cure, ameliorate, stabilize or prevent a disease, condition, or disorder.

The term “C_{x-y}” when used in conjunction with a chemical moiety, such as alkyl, alkenyl, or alkynyl is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond respectively. The term —C_{x-y}alkenylene-refers to a substituted or unsubstituted alkenylene chain with from x to y carbons in the alkenylene chain. For example, —C₂₋₆alkenylene-may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene-refers to a substituted or unsubstituted alkynylene chain with from x to y carbons in the alkynylene chain. For example, —C₂₋₆alkynylene-may be selected from ethynylene, propynylene, butynylene, pentynylene, and hexynylene, any one of which is optionally substituted. An alkynylene chain may have one triple bond or more than one triple bond in the alkynylene chain.

“Heteroalkylene” refers to a straight divalent hydrocarbon chain including at least one heteroatom in the chain, containing no unsaturation, and preferably having from one to twelve carbon atoms and from one to 6 heteroatoms, e.g., —O—, —NH—, —S—. The heteroalkylene chain is attached to the rest of the molecule through a single bond and to the radical group through a single bond. The points of attachment of the heteroalkylene chain to the rest of the molecule and to the radical group are through the terminal atoms of the chain. In other embodiments, a heteroalkylene comprises one to five carbon atoms and from one to three heteroatoms. In other embodiments, a heteroalkylene comprises one to four carbon atoms and from one to three heteroatoms. In other embodiments, a heteroalkylene comprises one to three carbon atoms and from one to two heteroatoms. In other embodiments, a heteroalkylene comprises one to two carbon atoms and from one to two heteroatoms. In other embodiments, a heteroalkylene comprises one carbon atom and from one to two heteroatoms. In other embodiments, a heteroalkylene comprises five to eight carbon atoms and from one to four heteroatoms. In other embodiments, a heteroalkylene comprises two to five carbon atoms and from one to three heteroatoms. In other embodiments, a heteroalkylene comprises three to five carbon atoms and from one to three heteroatoms. Unless stated otherwise specifically in the specification, a heteroalkylene chain is optionally substituted by one or more substituents such as those substituents described herein.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, and 6-6 fused ring systems. Exemplary carbocycles include cyclopentyl, cyclohexyl, cyclohexenyl, adamantyl, phenyl, indanyl, and naphthyl.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, and 6-6 fused ring systems.

The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also include polycyclic ring systems having two or more cyclic rings in which two or more carbons are common to two adjoining rings wherein at least one of the rings is heteroaromatic, e.g., the other cyclic rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH),

hydrazine(═NNH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═NNH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH),
hydrazine(═NNH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or
2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants, unless specified otherwise.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

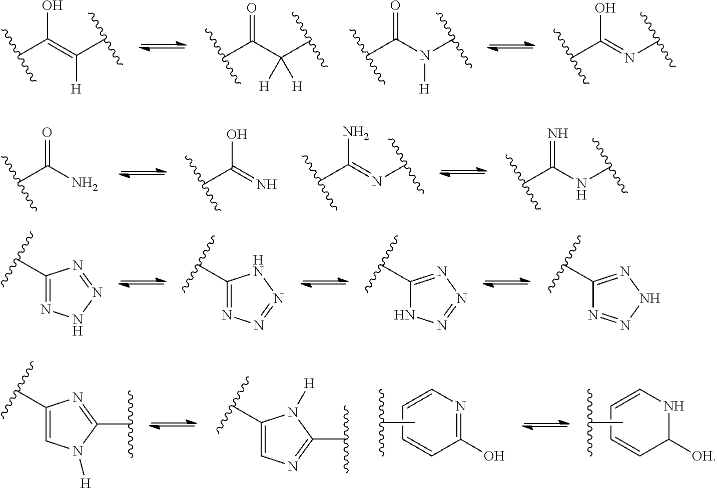

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made, for example, by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (2H), tritium (3H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³S, ³⁶S, ³⁵Ci, ³⁷Cl, ⁷⁹Br, ⁸¹Br, and/or ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds can be synthesized, for example, using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Disclosed herein are compositions and methods useful for treating a disease or condition. The compositions and methods herein can be used for promotion of myelination in disease states, such as neurological disease states or chemotherapy-related cognitive impairment. The compositions and methods provided herein comprise Tropornyosin receptor kinase B (TrkB) agonists and uses thereof.

### Troponyosin Receptor Kinase B (TrkB) Agonists

Tropomyosin receptor kinase B (TrkB), also known as tyrosine receptor kinase B, or BDNF/NT-3 growth factors receptor or neurotrophic tyrosine kinase, receptor, type 2 is a protein that, in humans, is encoded by the NTRK2 gene. TrkB is a receptor for brain-derived neurotrophic factor (BDNF). TrkB can be a high affinity catalytic receptor for several neurotrophins, which can be small protein growth factors that can promote the survival and differentiation of distinct cell populations. In some aspects, the neurotrophins that can activate TrkB are: BDNF (Brain Derived Neurotrophic Factor), neurotrophin-4 (NT-4), neurotrophin-3 (NT-3), and combinations thereof. As such, TrkB can mediate the multiple effects of these neurotrophic factors, such as neuronal differentiation and survival. In some aspects, activation of the TrkB receptor can lead to down-regulation of the KCC2 chloride transporter in cells of the central nervous system (CNS). The TrkB receptor is part of a large family of receptor tyrosine kinases. Currently, there are three TrkB isoforms in the mammalian CNS. The full-length isoform (TK+) is a typical tyrosine kinase receptor, and transduces the BDNF signal via Ras-ERK, PI3K, and PLCγ. By contrast, two truncated isoforms (TK−: T1 and T2) possess the same extracellular domain, transmembrane domain, and first 12 intracellular amino acid sequences as TK+. However, the C-terminal sequences can be isoform-specific (11 and 9 amino acids, respectively). T1 can be involved in the regulation of cell morphology and calcium influx. The TrkB receptor is part of a large family of receptor tyrosine kinases. A “tyrosine kinase” is an enzyme which is capable of adding a phosphate group to certain tyrosines on target proteins, or “substrates”. A receptor tyrosine kinase is a “tyrosine kinase” which is located at the cellular membrane, and is activated by binding of a ligand to the receptor's extracellular domain. Other examples of tyrosine kinase receptors include the insulin receptor, the IGF1 receptor, the MuSK protein receptor, the Vascular Endothelial Growth Factor (or VEGF) receptor, etc.

TrkB contains several domains, including a ligand-binding site in the extracellular portion, a single-transmembrane a helix, a juxtamembrane region, and an intracellular tyrosine kinase domain. Homodimers of BDNF, NT3, or NT4 induce TrkB dimerization and kinase activation through a series of autophosphorylation events at Tyr515, Tyr705-706, and Tyr816. SHC1 and other signaling and adaptor proteins are recruited to TrkB that is phosphorylated at Tyr515 (pTyr515) and trigger signaling through the RAS-ERK (RAS-extracellular signal-regulated kinase) and PI3K (phosphatidylinositol 3-kinase)-AKT cascades. Phosphorylation of TrkB at Tyr816 elicits activation of PLCγ (phospholipase C-γ), which triggers PKC (protein kinase C)-Ca2+ signaling. These pathways collectively account for anti-apoptotic signaling, local protein synthesis, and gene regulation that ultimately lead to increased neurogenesis, neuronal growth and differentiation, synaptogenesis, synaptic plasticity, and other physiological effects of BDNF and other neurotrophic factors. In some aspects, Zn2+ transactivates TrkB through an indirect mechanism in which Zn2+ ions inhibit the kinase CSK (C-terminal SRC kinase), thus preventing it from inhibiting the kinase SRC. Disinhibited SRC then phosphorylates TrkB at Tyr706-707, which in turn activates the TrkB kinase domain, leading to phosphorylation of other tyrosine residues required for signaling through the ERK and AKT pathways. SRC and TrkB also mutually activate each other. In some aspects, K252a can be a compound that inhibits TrkB by binding to the tyrosine kinase domain. In some aspects, an agonist provided herein can be a BDNF mimetic. In some aspects, a composition provided herein can activate, modulate, inhibit, or otherwise act on any one of: Deprenyl, amitriptyline, DMAQ-B1, 7, 8-DHF, LM22A-4, Deoxygedunin, RAP, RAS, c-RAF, b-RAF, MEK, ERK, PI3K, PDK1, AKT, SOS, SHC1, GRB1, FRS2, GAB1, K252a, SRC, CSK, IP3, DAG, CAMK, PKC, derivatives thereof, mimetics thereof, modified versions thereof, and any combination thereof.

In some embodiments, a TrkB agonist of the disclosure is selected from: N-Acetylserotonin (also known as normelatonin), Amitriptyline (IUPAC name: 3-(10,11-dihydro-5H-dibenzo[a,d]cycloheptene-5-ylidene)-N,N-dimethylpropan-1-amine), BNN-20 (also known as 170-spiro-(androst-5-en-17,2′-oxiran)-3β-ol), BNN-27 (also known as 17α,20R-epoxypregn-5-ene-3β,21-diol), Brain-derived neurotrophic factor (BDNF), Deoxygedunin, 7,8-Dihydroxyflavone, 4′-Dimethylamino-7,8-dihydroxyflavone, Diosmetin (also known as 5,7,3′-trihydroxy-4′-methoxyflavone), HIOC (IUPAC name: N-[2-(5-Hydroxy-1H-indol-3-yl)ethyl]-2-oxo-3-piperidinecarboxamide),

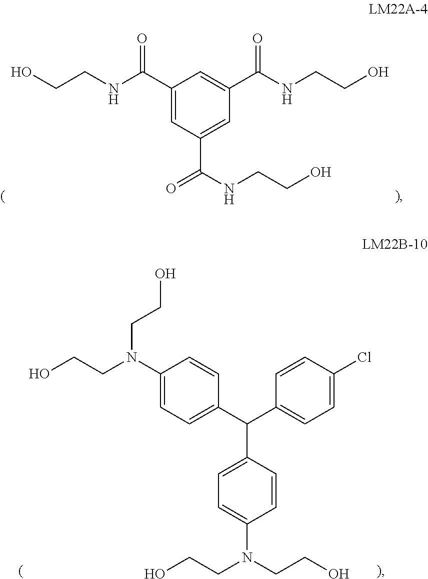 [SMILES: O=C(NCCO)c1cc(C(=O)NCCO)cc(C(=O)NCCO)c1 ; OCCN(CCO)c1ccc(C(c2ccc(Cl)cc2)c2ccc(N(CCO)CCO)cc2)cc1]

Neurotrophin-3, Neurotrophin-4, Norwogonin, R7 (IUPAC name: [8-(Dimethylcarbamoyloxy)-4-oxo-2-phenylchromen-7-yl] N,N-dimethylcarbamate), R13 (other name: 4-Oxo-2-phenyl-4H-chromene-7,8-diyl bis(methylcarbamate)), and 7,8,3′-Trihydroxyflavone (IUPAC name: 7,8-Dihydroxy-2-(3-hydroxyphenyl)chromen-4-one).

In some embodiments, a TrkB agonist provided herein is represented by Formula (II):

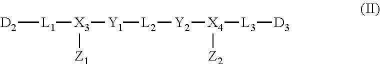

wherein:
L₁ and L₃ are independently selected from the group comprising C₁-C₅ alkylene, arylene, aralkylene, and substituted arylene;
L₂ is selected from the group consisting of C₁-C₅ alkylene, arylene, aralkylene, and a substituted arylene,

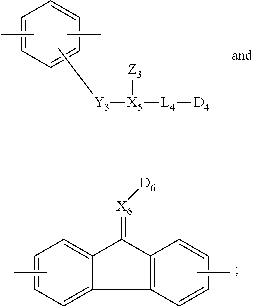

L₄ is C₁-C₅ alkylene;
Z₁, Z₂, and Z₃ are independently selected from the group consisting of H, alkyl, aryl, and aralkyl;
X₃, X₄, X₅, and X₆ are independently N or CH;
Y₁, Y₂, and Y₃ are independently carbonyl, sulfonyl, or methylene; and D₂, D₃, D₄, and D₅ are independently selected from H, alkyl, halo, hydroxyl, mercapto, mercaptoalkyl, alkoxyl, aryloxyl, aralkoxyl, acyloxyl, carboxyl, alkyloxycarbonyl, aryloxycarbonyl, aralkoxycarbonyl, acylamino, carbamoyl, alkylcarbamoyl, dialkylcarbamoyl,

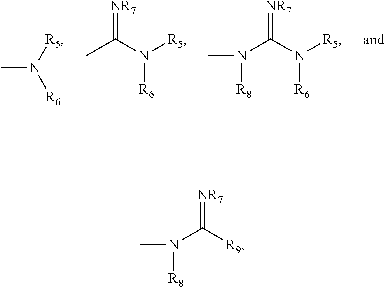

wherein R₅, R₆, R₇, R₈, and R₉ can be independently selected from H, alkyl, aralkyl, and aryl; or a pharmaceutically acceptable salt thereof.

In some embodiments, L₁ and L₃ are independently selected from C₁-C₅ alkylene and substituted arylene. In some embodiments, L₁ and L₃ are independently selected from C₁-C₃ alkylene, such as methylene, ethylene, or propylene. In some embodiments, L₁ and L₃ are both C₁-C₃ alkylene. In some embodiments, L₁ and L₃ are both ethylene.

In some embodiments, L₂ is selected from the group consisting of C₁-C₅ alkylene,

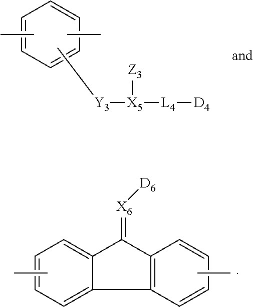

In some embodiments, L₂ is

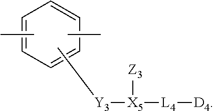

In some embodiments, L₄ is C₁-C₃ alkylene, such as methylene, ethylene, or propylene. In some embodiments, L₄ is ethylene.

In some embodiments, Z₁, Z₂, and Z₃ are independently selected from the group consisting of H, and alkyl. In some embodiments, at least one of Z₁, Z₂, and Z₃ is H. In some embodiments, Z₁, Z₂, and Z₃ are each H.

In some embodiments, X₃, X₄, X₅, and X₆ are each N. In some embodiments, Y₁, Y₂, and Y₃ are independently carbonyl, or sulfonyl. In some embodiments, Y₁, Y₂, and Y₃ are each carbonyl. In some embodiments, Y₁, Y₂, and Y₃ are each sulfonyl.

In some embodiments, D₂, D₃, D₄, and D₅ are independently selected from H, alkyl, halo, hydroxyl, alkoxyl, aryloxyl, carboxyl,

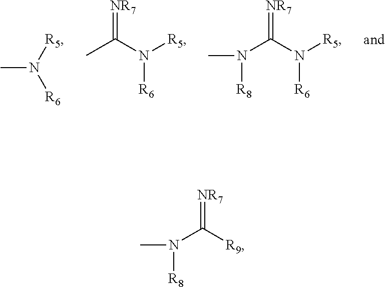

wherein R₅, R₆, R₇, R₈, and R₉ are independently selected from H, alkyl, aralkyl, and aryl;

or a pharmaceutically acceptable salt thereof. In some embodiments, D₂, D₃, D₄, and D₅ are independently selected from hydroxyl and

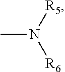

wherein R₅, and R₆ are independently selected from H and alkyl.

In some embodiments, the compound is selected from the group consisting of

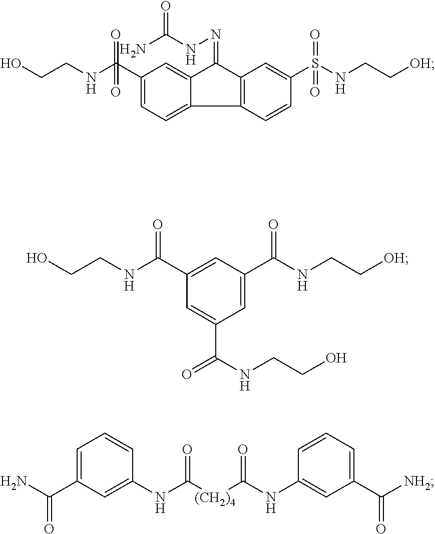

and a pharmaceutically acceptable salt of any one thereof.

In some embodiments, the compound is

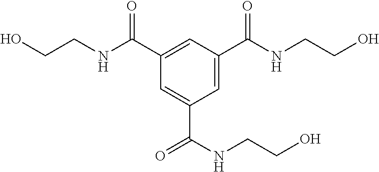 [SMILES: O=C(NCCO)c1cc(C(=O)NCCO)cc(C(=O)NCCO)c1]

(LM22A-4) or a pharmaceutically acceptable salt thereof. In some embodiments for the methods disclosed herein, he method further comprises administering a second therapeutic agent selected from an agent that promotes myelination. In some embodiments, the second therapeutic agent is selected from a parasympathomimetic drug, a Glycogensynthase kinase 3 beta inhibitor and a sterol.

A TrkB agonist can be a biologically active agent, such as any ligand, protein, peptide, small molecule or peptidomimetic which activates TrkB. In some aspects, a TrkB agonist also activates TrkB signaling of the TrkB receptor. Examples of TrkB agonists can include, but are not limited to, LM22A-1 (5-oxo-1-prolyl-1-histidyl-1-tryptophan methyl ester), LM22A-2 (2-[2,7-bis[[(2-hydroxyethyl)amino]sulfonyl]-9H-fluoren-9-ylidene]-hydrazinecarboxamide), LM22A-3 (N-[4-[2-[5-amino-4-cyano-1-(2-hydroxy ethyl)-1H-pyrazol-3-yl]-2-cyanoethenyl]phenyl]-acetamide), and LM22A-4 (N,N′,N″-tris(2-hydroxyethyl)-1,3,5-benzenetricarboxamide), neurotrophin-3 (NT-3), neurotrophin-4 (NT-4), N-acetylserotonin, N-(2-(5-hydroxy-1H-indol-3-yl)ethyl)-2-oxopiperideine-3-carboximide (HIOC), amitriptyline, 7,8-dihydroxyflavone, 7,8,3′-trihydroxyflavone, 4′-dimethylamino-7,8-dihydroxyflavone, and deoxygedunin. In some aspects an agonist of TrkB can be selected from the group comprising: 3,7-Dihydroxyflavone; 3,7,8,2′-Tetrahydroxyflavone; 4′-Dimethylamino-7,8-dihydroxyflavone (4′-DMA-7,8-DHF); 7,3-Dihydroxyflavone; 7,8-Dihydroxyflavone (7,8-DHF); 7,8,2′-Trihydroxyflavone; 7,8,3′-Trihydroxyflavone; Amitriptyline; BNN-20; Brain-derived neurotrophic factor (BDNF); Deoxygedunin; Deprenyl; Diosmetin; DMAQ-B1; HIOC; LM22A-4; N-Acetylserotonin (NAS); Neurotrophin-3 (NT-3); Neurotrophin-4 (NT-4); Norwogonin (5,7,8-THF); R7 (prodrug of 7,8-DHF); R13 (prodrug of 7,8-DHF); TDP6; and combinations thereof.

In some aspects, an agonist provided herein can be: 7,8-dihydroxyflavone (7,8-DHF), deoxygedunin, LM22A-4, demethylasterriquinone B1 (DMAQ-B1), amitriptyline, and deprenyl.

In some aspects, an agonist provided herein can activate TrkA, TrkB, and TrkC. In some aspects, an agonist provided herein can reduce apoptosis in a brain-derived cell line overexpressing TrkB. In some aspects, an agonist provided herein can bind an extracellular domain of TrkB as measured by surface plasmon resonance, other biochemical assays, and TrkB phosphorylation by Western blots, and combinations thereof. In some aspects, an agonist provided herein can promote axonal regeneration. In some aspects, an agonist can promote TrkB phosphorylation, neurite outgrowth in vitro, or combinations thereof.

TrkB agonists provided herein can be in the form of peptides having two or more amino acids. An amino acid can include the residues of the natural a-amino acids (e.g., Ala, Arg, Asn, Asp, Cys, Glu, Gin, Gly, His, Hyl, He, Leu, Met, Phe, Pro, Ser, Thr, Tip, Tyr, and Val) in D or L form, as well as R-amino acids, synthetic and unnatural amino acids. Many types of amino acid residues can be useful in a TrkB agonist composition. In some aspects, an amino acid may also include genetically-encoded amino acids. Examples of amino acids that can be utilized in the peptides described herein can be found, for example, in Fasman, 1989, CRC Practical Handbook of Biochemistry and Molecular Biology, CRC Press, Inc., and the reference cited therein. Another source of a wide array of amino acid residues is provided by the website of RSP Amino Acids LLC. A peptide can comprise a sequence of from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or more amino acid residues in which the a-carboxyl group of one amino acid is joined by an amide bond to the main chain (a- or β-) amino group of the adjacent amino acid. In some aspects, a peptide can be over 45 amino acids. The peptides provided herein for use in the described and claimed methods and compositions can be cyclic. TrkB agonists which are peptides, polypeptides, and/or proteins (including functional portions and functional variants thereof) can be obtained by methods known in the art. Suitable methods of de novo synthesizing polypeptides and proteins are described in references, such as Chan et al, Fmoc Solid Phase Peptide Synthesis. Oxford University Press, Oxford, United Kingdom, 2005; Peptide and Protein Drug Analysis, ed. Reid, R., Marcel Dekker, Inc., 2000; Epitope Mapping, ed. Westwoood et al, Oxford University Press, Oxford, United Kingdom, 2000; and U.S. Pat. No. 5,449,752. Also, polypeptides and proteins can be recombinantly produced using the nucleic acids described herein using standard recombinant methods. See, for instance, Sambrook et al, Molecular Cloning: A Laboratory Manual. 3^{rd} ed., Cold Spring Harbor Press, Cold Spring Harbor, N.Y. 2001; and Ausubel et al, Current Protocols in Molecular Biology. Greene Publishing Associates and John Wiley & Sons, N Y, 1994. Methods of isolation and purification are well-known in the art. Alternatively, TrkB agonists, such as polypeptides, and/or proteins provided herein (including functional portions and functional variants thereof) can be commercially synthesized by companies.

An effective amount for a subject will depend upon the subject's body weight, size, and health; the nature and extent of the condition; and the therapeutic selected for administration. An effective amount for a given situation can be determined by routine experimentation that may be within the skill and judgment of a clinician. An effective amount, as used herein, can refer to an amount of an agonist composition sufficient to produce a measurable biological response. Actual dosage levels of the delivery vehicle composition can be varied so as to administer an amount that may be effective to achieve the desired response for a particular subject and/or application. The selected dosage level will depend upon a variety of factors including the type of tissue being addressed, the types of cells, combination with other drugs or treatments, severity of the condition being treated, and the physical condition and prior medical history of the subject being treated. Preferably, a minimal dose can be administered, and a dose can be escalated in the absence of dose-limiting toxicity to a minimally effective amount.

In some aspects, a method provided herein comprises promoting or maintaining growth of a nervous system cell. A nervous system cell can be selected from the group comprising: neurons, dendrites, glia, oligodendrocytes, Schwann, and combinations thereof. In some aspects, a method provided herein comprises promoting or maintaining growth of glial cells. In some aspects, a method provided herein comprises promoting or maintaining growth of oligodendrocytes. In some aspects, a method provided herein comprises promoting growth of a nervous system cell wherein the growth is improved from about 1%, 2%, 3%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 20%, or up to 25% over that of a comparable method absent an agonist provided herein. In some aspects, a method provided herein comprises delaying or preventing death of a nervous system cell provided herein.

### Cognitive Dysfunction

Provided herein can be compositions and methods for treating cognitive dysfunction. In some aspects, the present disclosure provides a method of preventing or treating a cancer therapeutic-related cognitive dysfunction, comprising administering to a subject in need thereof an effective amount of a TrkB agonist, wherein said TrkB agonist is administered to the subject before, during, or after completion of the administration of a cancer treatment regimen or a combination thereof. In some aspects, cognitive dysfunction can comprise being unusually disorganized, confusion, difficulty concentrating, difficulty finding the right word, difficulty learning new skills, difficulty multitasking, fatigue, feeling of mental fogginess, short attention span, short-term memory problems, taking longer than usual to complete routine tasks, trouble with verbal memory, adynamia, lack of motivation, impulsiveness, disinhibition, verbosity, tangentiality, irritability, fidgety, low frustration tolerance, trouble with visual memory, and combinations thereof. In some aspects, cognitive dysfunction can comprise short term memory problems. Examples of cognitive dysfunction can comprise trouble remembering the names of people, trouble remembering the flow of a conversation, increased tendency to misplace things, and combinations thereof. In some aspects, a cognitive impairment can also be selected from the group comprising: Parkinson's disease, Alzheimer's disease, Huntington's disease, Cushing's disease, Lewy body disease, multiple sclerosis, stroke, addictive disorders (for example smoking, drug abuse, cocaine dependence, gambling and other impulse control effects), pervasive, development disorder, autism, fragile X syndrome, anxiety disorders (e.g. acute and chronic panic, post-traumatic stress disorder, generalized anxiety disorder), Prader-Willi syndrome, schizophrenia unassociated with aggression, bipolar disorder, depression, vascular dementia, mild cognitive impairment, dementia, amnestic disorders, delirium, and other cognitive impairments. In some embodiments, the cognitive dysfunction comprises short-term memory problems.

In some aspects, a method provided herein can comprise administering a test of cognitive function. A test of cognitive function can be administered, before, during, and/or after administration of an effective amount of an agonist, such as a TrkB agonist provided herein. In some embodiments, the methods disclosed herein comprise administering to the subject a test of cognitive function prior to administration of the TrkB agonist. In some embodiments, the test of cognitive function indicates that the subject has a cognitive dysfunction. In other embodiments, the test of cognitive function indicates that the subject does not have a cognitive dysfunction. In some aspects, a test of cognitive function can be administered after administration of an effective amount of a TrkB agonist wherein the agonist indicates maintenance or improvement in a cognitive function of a subject. In some aspects, a test of cognitive function administered after administration of an effective amount of a TrkB agonist can indicate improvement in one or more symptoms of cognitive dysfunction of a subject. In some aspects, a test of cognitive function can be selected from the General Practitioner Assessment of Cognition (GPCOG), Memory Impairment Screen (MIS), MiniMental State Exam (MMSE), and the Six Item Cognitive Impairment Test (6CIT).

In some aspects, a test of cognitive function can be administered after administration of an effective amount of an agonist, such as a TrkB agonist. In some aspects, a test of cognitive function can be administered after administration of an effective amount of an agonist, such as a TrkB agonist and can indicate maintenance or improvement in cognitive function of a subject. In some aspects, a test of cognitive function administered after administration of an effective amount of a TrkB agonist can indicate improvement in one or more symptoms of cognitive dysfunction of a subject. In some aspects, improvement can be elimination of a cognitive dysfunction. In some aspects, improvement can be from about 1%, 2%, 3%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40% as measured by an assessment of a symptom of cognitive dysfunction.

In some aspects, the present disclosure provides a method of improving cognition in a healthy subject, comprising administering to said healthy subject an effective amount of a TrkB agonist, wherein healthy subject does not suffer from a cognitive disease or disorder.

### Cancer Therapeutic

In some aspects, provided herein can be a cancer therapeutic. A cancer therapeutic may be administered before, during, or after an agonist provided herein. In some aspects, a composition or method provided herein can be administered to a subject comprising a cancer. In some aspects, a composition or method provided herein can be administered to a subject with: breast cancer, ovarian cancer, colon cancer, rectal cancer, bladder cancer, epithelial cancer, bone cancer, brain cancer, breast cancer, esophageal cancer, gastrointestinal cancer, leukemia, liver cancer, lung cancer, lymphoma, myeloma, ovarian cancer, prostate cancer, sarcoma, stomach cancer, thyroid cancer, acute lymphocytic cancer, acute myeloid leukemia, alveolar rhabdomyosarcoma, anal canal, ocular cancer, cancer of the neck, gallbladder cancer, pleural cancer, oral cancer, cancer of the vulva, colon cancer, cervical cancer, fibrosarcoma, gastrointestinal carcinoid tumor, Hodgkin lymphoma, kidney cancer, mesothelioma, mastocytoma, melanoma, multiple myeloma, nasopharynx cancer, non-Hodgkin lymphoma, pancreatic cancer, peritoneal cancer, renal cancer, sarcoma, skin cancer, small intestine cancer, soft tissue cancer, solid tumors, stomach cancer, testicular cancer, thyroid cancer, and combinations thereof. In some embodiments, the subject has a cancer selected from breast cancer, ovarian cancer, prostate cancer, leukemia, lymphoma, brain tumor, and sarcoma. In some embodiments, the subject has a cancer selected from a cancer of the central nervous system. In some embodiments, the subject has a cancer selected from a cancer other than cancer of the central nervous system.

In some aspects, a subject may have a predisposition to a cancer. A predisposition may be genetic or non-genetic. In some aspects, a predisposition may be environmental. In some aspects, a predisposition may be a genetic predisposition to cancer. In some embodiments, the genetic predisposition to cancer is a genetic predisposition to breast cancer, ovarian cancer, colon cancer, ectum cancer, leukemia, lymphoma, brain tumor, or sarcoma. A genetic predisposition to cancer may comprise a mutation in a gene. In some aspects, a genetic predisposition may comprise a mutation in p53, HER2, BRCA1, BRCA2, a RAS family, or any combination thereof. In some aspects, a genetic predisposition to cancer comprises a family history of cancer.

In some aspects, a subject may be absent: Alzheimer's disease, Huntington's disease, amyotrophic lateral sclerosis, Rett syndrome, epilepsy, Parkinson's disease, spinal cord injury, stroke, hypoxia, ischemia, brain injury, diabetic neuropathy, peripheral neuropathy, nerve transplantation complications, motor neuron disease, multiple sclerosis, HIV dementia, peripheral nerve injury, hearing loss, depression, obesity, metabolic syndrome, pain, and cancer. In some aspects, a subject may comprise cognitive impairment or dysfunction but may not comprise a disease selected from: Alzheimer's disease, Huntington's disease, amyotrophic lateral sclerosis, Rett syndrome, epilepsy, Parkinson's disease, spinal cord injury, stroke, hypoxia, ischemia, brain injury, diabetic neuropathy, peripheral neuropathy, nerve transplantation complications, motor neuron disease, multiple sclerosis, HIV dementia, peripheral nerve injury, hearing loss, depression, obesity, metabolic syndrome, pain, and cancer.

In some aspects, a cancer therapeutic comprises a chemotherapeutic agent or compound. A chemotherapeutic agent or compound can be a chemical compound useful in the treatment of cancer. The chemotherapeutic cancer agents that can be, but are not limited to, mitotic inhibitors (vinca alkaloids). These include vincristine, vinblastine, vindesine and Navelbine™ (vinorelbine, 5′-noranhydroblastine). In yet other cases, chemotherapeutic cancer agents include topoisomerase I inhibitors, such as camptothecin compounds. As used herein, “camptothecin compounds” include CAMPTOSAR™ (irinotecan HCL), HYCAMTIN™ (topotecan HCL) and other compounds derived from camptothecin and its analogues. Another category of chemotherapeutic cancer agents that can be used in the methods and compositions disclosed herein are podophyllotoxin derivatives, such as etoposide, teniposide and mitopodozide. The present disclosure further encompasses other chemotherapeutic cancer agents known as alkylating agents, which alkylate the genetic material in tumor cells. These include without limitation cisplatin, cyclophosphamide, nitrogen mustard, trimethylene thiophosphoramide, carmustine, busulfan, chlorambucil, belustine, uracil mustard, chlomaphazin, and dacarbazine. The disclosure encompasses antimetabolites as chemotherapeutic agents. Examples of these types of agents include cytosine arabinoside, fluorouracil, methotrexate, mercaptopurine, azathioprime, and procarbazine. An additional category of chemotherapeutic cancer agents that may be used in the methods and compositions disclosed herein include antibiotics. Examples include without limitation doxorubicin, bleomycin, dactinomycin, daunorubicin, mithramycin, mitomycin, mytomycin C, and daunomycin. There are numerous liposomal formulations commercially available for these compounds. The present disclosure further encompasses other chemotherapeutic cancer agents including without limitation anti-tumor antibodies, dacarbazine, azacytidine, amsacrine, melphalan, ifosfamide and mitoxantrone.

The compositions provided herein can be administered in combination with other anti-tumor agents, including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents can be mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents can also be used. Suitable anti-angiogenic agents for use before, during or after, the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed engineered cells can include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; avastin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; erythrocyte gene therapy; velaresol; veramine; verdins; verteporfin; vinorelbine; vinxaltine; vitaxin; vorozole; zanoterone; zeniplatin; zilascorb; and zinostatin stimalamer. Any of the aforementioned chemotherapeutics can be administered at a clinically effective dose. A chemotherapeutic can also be administered from about day: −12 months, −6 months, −4 months, −2 months, −1 month, −15, −14, −13, −12, −11, −10, −9, −8, −7, −6, −5, −4, −3, −2, −1, 0, 1, 2, 3, 4, 5, 6, 7, 8, 9, 10, 11, 12, 13, or up to about day 14 after administration of an agonist provided herein.

In some aspects, a cancer therapeutic may comprise radiation or radiotherapy. Radiation may comprise external beam radiation therapy whereby radiation is localized to a cancer lesion. In some aspects, radiation may comprise internal radiation therapy. Internal radiation therapy is a treatment in which a source of radiation is put inside your body. The radiation source can be solid or liquid. Whole body radiation may be administered at 12 Gy. A radiation dose may comprise a cumulative dose of 12 Gy to the whole body, including healthy tissues. A radiation dose may comprise from 5 Gy to 20 Gy. A radiation dose may be 5 Gy, 6 Gy, 7 Gy, 8 Gy, 9 Gy, 10 Gy, 11 Gy, 12, Gy, 13 Gy, 14 Gy, 15 Gy, 16 Gy, 17 Gy, 18 Gy, 19 Gy, or up to 20 Gy. Radiation may be whole body radiation or partial body radiation. In the case that radiation is whole body radiation it may be uniform or not uniform. For example, when radiation may not be uniform, narrower regions of a body such as the neck may receive a higher dose than broader regions such as the hips.

In some aspects, a cancer therapeutic comprises an immunomodulatory therapy. In some cases, an immunostimulant can be administered to a subject. An immunostimulant can be specific or non-specific. A specific immunostimulant can provide antigenic specificity such as a vaccine or an antigen. A non-specific immunostimulant can augment an immune response or stimulate an immune response. A non-specific immunostimulant can be an adjuvant. Immunostimulants can be vaccines, colony stimulating agents, interferons, interleukins, viruses, antigens, co-stimulatory agents, immunogenicity agents, immunomodulators, or immunotherapeutic agents. An immunostimulant can be a cytokine such as an interleukin. One or more cytokines can be introduced with cells of the invention. Cytokines can be utilized to boost cytotoxic T lymphocytes (including adoptively transferred tumor-specific cytotoxic T lymphocytes) to expand within a tumor microenvironment. In some aspects, a cytokine can be administered to a subject such as IL-2, IL-7, IL-12, IL-15, IL-21, or any combination thereof. In some aspects, an immunomodulatory therapy can comprise an immunotherapy. Immunotherapy can be a checkpoint inhibitor, adoptive cell transfer, monoclonal antibodies or portions thereof, vaccines, and any combination thereof.

In some cases, any number of administrations of a cancer therapeutic may be applied to a subject. For example, from 0, 1, 2, 3, 4, 5, 6, 7, 8, 9, 10, or more administrations of a cancer therapeutic may be administered to a subject. In some aspects, at least 1 or 2 administrations of a cancer therapeutic, such a chemotherapy or radiation, are given to a subject.

In some embodiments for the methods disclosed herein, the cancer treatment regimen comprises one or more doses of chemotherapy. In some embodiments, the cancer treatment regimen comprises one or more doses of radiation therapy. In some embodiments, the cancer treatment regimen comprises one or more doses of immunomodulatory therapy.

In some aspects, a method provided herein comprises administering a TrkB agonist to a subject after completion of an administration of a cancer treatment regimen. In some aspects, a method provided herein comprises administering a TrkB agonist to a subject for about 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or up to about 10 years after completion of the administration of the cancer treatment regimen. In some aspects, a method provided herein comprises administering a TrkB agonist to a subject for about 1 month to about 2 months, from about 2 months to about 3 months, from about 3 months to about 4 months, from about 4 months to about 5 months, from about 5 months to about 6 months, from about 6 months to about 7 months, from about 7 months to about 8 months, from about 8 months to about 9 months, from about 9 months to about 10 months, from about 10 months to about 11 months, from about 11 months to about 1 year after completion of the administration of the cancer treatment regimen. In some aspects, a method provided herein comprises administering a TrkB agonist to a subject from about 1 month to about 5 months, from about 1 month to about 8 months after complement of the administration of the cancer treatment regimen. In some aspects, a method provided herein comprises administering a TrkB agonist to a subject before the administration of the cancer treatment regimen. In some aspects, a TrkB agonist can be administered to a subject for about 1 day to about 3 months before the administration of the cancer treatment regimen. In some aspects, a TrkB agonist can be administered to a subject for about 1 day to about 1 month before the administration of the cancer treatment regimen. In some aspects, a TrkB agonist can be administered to a subject for about 1 day to about 2 months before the administration of the cancer treatment regimen. In some aspects, a method provided herein comprises administering a TrkB agonist to a subject during or concurrent with a cancer treatment regimen. In some aspects, a method provided herein comprises administering an agonist, such as a TrkB agonist to a subject within 10, 9, 8, 7, 6, 5, 4, 3, 2, 1, 0.5 hours of administration of a dose of a cancer treatment regimen. In some aspects, a TrkB agonist can be administered to a subject within three hours of administration of a dose of the cancer treatment regimen. In some aspects, a TrkB agonist can be administered concurrently with a dose of a cancer treatment regimen. In some aspects, a TrkB agonist can be administered daily, every other day, every third day, every fourth day, every fifth day, every sixth day, weekly, bi-weekly, monthly, bi-monthly, yearly, biyearly, and any combination thereof.

### Pharmaceutical Compositions and Formulations

The compositions described throughout can be formulation into a pharmaceutical medicament and be used to treat a human or mammal, in need thereof, diagnosed with a disease or condition, particularly in tissues and cells that are associated with a layer of mucus through which the therapeutic agent must be delivered. Medicaments can be co-administered with any additional therapy.

In some aspects, the present disclosure provides a pharmaceutical formulation, comprising:

(a) a compound of Formula (II):

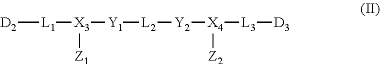

wherein:

L₁ and L₃ are independently selected from the group consisting of C₁-C₅ alkylene, arylene, aralkylene, and substituted arylene;

L₂ is selected from the group consisting of C₁-C₅ alkylene, arylene, aralkylene, substituted arylene,

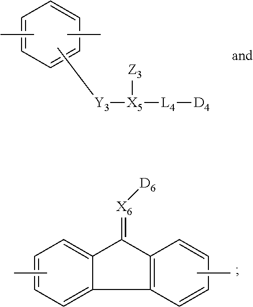

L₄ is C₁-C₅ alkylene;

Z₁, Z₂, and Z₃ are independently selected from the group consisting of H, alkyl, aryl, and aralkyl;
X₃, X₄, X₅, and X₆ are independently N or CH;

Y₁, Y₂, and Y₃ are independently carbonyl, sulfonyl, or methylene; and

D₂, D₃, D₄, and D₅ are independently selected from H, alkyl, halo, hydroxyl, mercapto, mercaptoalkyl, alkoxyl, aryloxyl, aralkoxyl, acyloxyl, carboxyl, alkyloxycarbonyl, aryloxycarbonyl, aralkoxycarbonyl, acylamino, carbamoyl, alkylcarbamoyl, dialkylcarbamoyl,

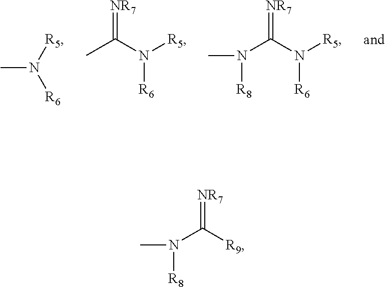

wherein R₅, R₆, R₇, R₅, and R₉ are independently selected from H, alkyl, aralkyl, and aryl;

or a pharmaceutically acceptable salt thereof,
(b) a second therapeutic agent; and
(c) a pharmaceutically acceptable excipient.

In some embodiments, the compound of Formula (II) is selected from the group consisting of:

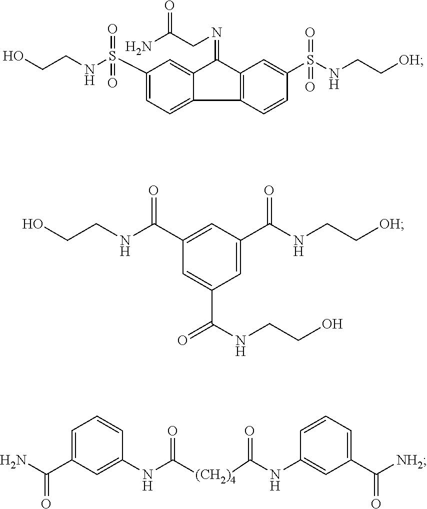

and a pharmaceutically acceptable salt of any one thereof.

In some embodiments, the second therapeutic agent is selected from a parasympathomimetic drug, a Glycogensynthase kinase 3 beta inhibitor, and a sterol.

For oral administration, an excipient may include pharmaceutical grades of mannitol, lactose, starch, magnesium stearate, sodium saccharine, talcum, cellulose, glucose, gelatin, sucrose, magnesium carbonate, and the like. If desired, a liposomal composition may also contain minor amounts of non-toxic auxiliary substances such as wetting agents, emulsifying agents, or buffers.

A composition provided herein can be administered orally, by subcutaneous or other injection, intravenously, intracerebrally, intramuscularly, parenterally, transdermally, nasally or rectally. The form in which the compound or composition is administered depends at least in part on the route by which the compound is administered. In some cases, a liposomal composition can be employed in the form of solid preparations for oral administration; preparations may be tablets, granules, powders, capsules or the like. In a tablet formulation, a composition is typically formulated with additives, e.g. an excipient such as a saccharide or cellulose preparation, a binder such as starch paste or methyl cellulose, a filler, a disintegrator, and other additives typically used in the manufacture of medical preparations. Methods for preparing such dosage forms may be apparent to those skilled in the art. An agonist composition may be in a pharmaceutically effective amount for therapeutic use in a biological system, including a patient or subject. A pharmaceutical composition may be administered daily or administered on an as needed basis. In certain embodiments, a pharmaceutical composition can be administered to a subject prior to bedtime. In some embodiments, a pharmaceutical composition can be administered immediately before bedtime. In some embodiments, a pharmaceutical composition can be administered within about two hours before bedtime, preferably within about one hour before bedtime. In another embodiment, a pharmaceutical composition can be administered about two hours before bedtime. In a further embodiment, a pharmaceutical composition can be administered at least two hours before bedtime. In another embodiment, a pharmaceutical composition can be administered about one hour before bedtime. In a further embodiment, a pharmaceutical composition can be administered at least one hour before bedtime. In a still further embodiment, a pharmaceutical composition can be administered less than one hour before bedtime. In still another embodiment, the pharmaceutical composition can be administered immediately before bedtime. A pharmaceutical composition is administered orally or rectally.

An appropriate dosage (“therapeutically effective amount”) of an active agent(s) in a composition may depend, for example, on the severity and course of a condition, a mode of administration, a bioavailability of a particular agent(s), the age and weight of a subject, a subject's clinical history and response to an active agent(s), discretion of a physician, or any combination thereof. A therapeutically effective amount of an active agent(s) in a composition to be administered to a subject can be in the range of about 100 μg/kg body weight/day to about 1000 mg/kg body weight/day whether by one or more administrations. In some embodiments, the range of each active agent administered daily can be from about 100 μg/kg body weight/day to about 50 mg/kg body weight/day, 100 μg/kg body weight/day to about 10 mg/kg body weight/day, 100 μg/kg body weight/day to about 1 mg/kg body weight/day, 100 μg/kg body weight/day to about 10 mg/kg body weight/day, 500 μg/kg body weight/day to about 100 mg/kg body weight/day, 500 μg/kg body weight/day to about 50 mg/kg body weight/day, 500 μg/kg body weight/day to about 5 mg/kg body weight/day, 1 mg/kg body weight/day to about 100 mg/kg body weight/day, 1 mg/kg body weight/day to about 50 mg/kg body weight/day, 1 mg/kg body weight/day to about 10 mg/kg body weight/day, 5 mg/kg body weight/dose to about 100 mg/kg body weight/day, 5 mg/kg body weight/dose to about 50 mg/kg body weight/day, 10 mg/kg body weight/day to about 100 mg/kg body weight/day, and 10 mg/kg body weight/day to about 50 mg/kg body weight/day.

As used herein, “pharmaceutically acceptable carrier” includes any and all solvents, dispersion media, coatings, antibacterial and antifungal agents, isotonic and absorption delaying agents, sweeteners, salts, buffers, and the like. Compositions provided herein can take the form of solutions, suspensions, emulsions, powders, sustained-release formulations, depots and the like. Examples of suitable carriers are described in “Remington's Pharmaceutical Sciences,” Martin. Such compositions will contain an effective amount of the biopolymer of interest, preferably in purified form, together with a suitable amount of carrier so as to provide the form for proper administration to the patient. As known in the art, the formulation will be constructed to suit the mode of administration. The pharmaceutically acceptable carriers may be prepared from a wide range of materials including, but not limited to, flavoring agents, sweetening agents and miscellaneous materials such as buffers and absorbents that may be needed in order to prepare a particular therapeutic composition. Buffering agents help to maintain the pH in the range which approximates physiological conditions. Buffers are preferably present at a concentration ranging from about 2 mM to about 50 mM. Suitable buffering agents for use with the instant invention include both organic and inorganic acids, and salts thereof, such as citrate buffers (e.g., monosodium citrate-disodium citrate mixture, citric acid-trisodium citrate mixture, citric acid-monosodium citrate mixture etc.), succinate buffers (e.g., succinic acid monosodium succinate mixture, succinic acid-sodium hydroxide mixture, succinic acid-disodium succinate mixture etc.), tartrate buffers (e.g., tartaric acid-sodium tartrate mixture, tartaric acid-potassium tartrate mixture, tartaric acid-sodium hydroxide mixture etc.), fumarate buffers (e.g., fumaric acid-monosodium fumarate mixture, fumaric acid-disodium fumarate mixture, monosodium fumarate-disodium fumarate mixture etc.), gluconate buffers (e.g., gluconic acid-sodium glyconate mixture, gluconic acid-sodium hydroxide mixture, gluconic acid-potassium gluconate mixture etc.), oxalate buffers (e.g., oxalic acid-sodium oxalate mixture, oxalic acid-sodium hydroxide mixture, oxalic acid-potassium oxalate mixture etc.), lactate buffers (e.g., lactic acid-sodium lactate mixture, lactic acid-sodium hydroxide mixture, lactic acid-potassium lactate mixture etc.) and acetate buffers (e.g., acetic acid-sodium acetate mixture, acetic acid-sodium hydroxide mixture etc.). Phosphate buffers, carbonate buffers, histidine buffers, trimethylamine salts, such as Tris, HEPES and other such known buffers can be used. Preservatives may be added to retard microbial growth, and may be added in amounts ranging from 0.2%-% (w/v). Suitable preservatives for use with the present invention include phenol, benzyl alcohol, m-cresol, octadecyldimethylbenzyl ammonium chloride, benzyaconium halides (e.g., chloride, bromide and iodide), hexamethonium chloride, alkyl parabens, such as, methyl or propyl paraben, catechol, resorcinol, cyclohexanol and 3-pentanol. Isotonicifiers are present to ensure physiological isotonicity of liquid compositions of the instant invention and include polhydric sugar alcohols, preferably trihydric or higher sugar alcohols, such as glycerin, erythritol, arabitol, xylitol, sorbitol and mannitol. Polyhydric alcohols can be present in an amount of between about 0.1% to about 25%, by weight, preferably 1% to 5% taking into account the relative amounts of the other ingredients. A carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride. Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, and combination thereof. Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimoniuni bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-lauryl-beta-iminodipropionate, myristoamphoacetate, lauryl betaine and lauryl sulfobetaine. The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s). The formulation is typically buffered to a pH of 3-8 for parenteral administration upon reconstitution. Suitable buffers include, but are not limited to, phosphate buffers, acetate buffers, and citrate buffers. Water soluble polymers can be often used in formulations for parenteral administration. Suitable water-soluble polymers include, but are not limited to, polyvinylpyrrolidone, dextran, carboxymethylcellulose, and polyethylene glycol.

The compositions described herein can be formulated under sterile conditions within a reasonable time prior to administration. The formulations to be used for in vivo administration must be sterile. That can be accomplished, for example, by filtration through sterile filtration membranes. For example, the formulations of the present invention may be sterilized by filtration. Therapeutic formulations of the product may be prepared for storage as lyophilized formulations or aqueous solutions by mixing the product having the desired degree of purity with optional pharmaceutically acceptable carriers, diluents, excipients or stabilizers typically employed in the art, i.e., buffering agents, stabilizing agents, preservatives, isotonifiers, non-ionic detergents, antioxidants and other miscellaneous additives, see Remington's Pharmaceutical Sciences, 16th ed., Osol, ed. (1980). Such additives are generally nontoxic to the recipients at the dosages and concentrations employed, hence, the excipients, diluents, carriers and so on are pharmaceutically acceptable. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols; amino acids, such as arginine, lysine, glycine, glutamine, asparagine, histidine, alanine, omithine, L-leucine, 2-phenylalanine, glutamic acid, threonine etc. ; organic sugars or sugar alcohols, such as lactose, trehalose, stachyose, arabitol, erythritol, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thiosulfate; low molecular weight polypeptides (i.e., <10 residues); proteins, such as human serum albumin, bovine serum albumin, gelatin or immunoglobulins; hydrophilic polymers, such as polyvinylpyrrolidone, saccharides, monosaccharides, such as xylose, mannose, fructose or glucose; disaccharides, such as lactose, maltose and sucrose; trisaccharides, such as raffinose; polysaccharides, such as, dextran and so on. Additional miscellaneous excipients include bulking agents, (e.g., starch), chelating agents (e.g., EDTA), antioxidants (e.g., ascorbic acid, methionine or vitamin E) and cosolvents. Non-ionic surfactants or detergents (also known as “wetting agents”) may be added to help solubilize the therapeutic agent, as well as to protect the therapeutic protein against agitation-induced aggregation, which also permits the formulation to be exposed to shear surface stresses without causing denaturation of the protein. Suitable non-ionic surfactants include polysorbates (20, 80 etc.), polyoxamers (184, 188 etc.), PLURONIC® polyols and polyoxyethylene sorbitan monoethers (TWEEN-20®, TWEEN-80® etc.). Non-ionic surfactants may be present in a range of about 0.05 mg/ml to about 1.0 mg/ml, preferably about 0.07 mg/ml to about 0.2 mg/ml. In some cases, a composition provided herein can comprise formulations, such as, liquid formulations having stability at temperatures found in a commercial refrigerator and freezer found in the office of a physician or laboratory, such as from about 20° C. to about 5° C., said stability assessed, for example, by microscopic analysis, for storage purposes, such as for about 60 days, for about 120 days, for about 180 days, for about a year, for about 2 years or more. The liquid formulations of the present invention also exhibit stability, as assessed, for example, by particle analysis, at room temperatures, for at least a few hours, such as one hour, two hours or about three hours prior to use. Examples of diluents include a phosphate buffered saline, buffer for buffering against gastric acid in the bladder, such as citrate buffer (pH 7.4) containing sucrose, bicarbonate buffer (pH 7.4) alone, or bicarbonate buffer (pH 7.4) containing ascorbic acid, lactose, or aspartame. Examples of carriers include proteins, e.g., as found in skim milk, sugars, e.g., sucrose, or polyvinylpyrrolidone. Typically these carriers would be used at a concentration of about 0.1-90% (w/v) but preferably at a range of 1-50%.

In some cases, formulations can be presented in unit-dose or multi-dose containers, for example sealed ampoules and vials, and can be stored in a frozen or freeze-dried (lyophilized) condition requiring only the addition of sterile liquid carrier immediately prior to use. For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated in some cases. Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner. In some cases, compositions can also be formulated as a preparation for implantation or injection. Thus, for example, a composition can be formulated with suitable polymeric, aqueous, and/or hydrophilic materials, or resins, or as sparingly soluble derivatives (e.g., as a sparingly soluble salt). The compounds can also be formulated in rectal compositions, creams or lotions, or transdermal patches. A formulation can be an ocular formulation or a topical formation. Pharmaceutical formulations for ocular administration can be in the form of a sterile aqueous solution or suspension of particles formed from one or more polymer-drug conjugates. Acceptable solvents include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol. In still other embodiments, the delivery vehicle composition can be formulated for topical administration to mucosa. Suitable dosage forms for topical administration include creams, ointments, salves, sprays, gels, lotions, emulsions, liquids, and transdermal patches. The formulation may be formulated for transmucosal, transepithelial, transendothelial, or transdermal administration. The compositions contain one or more chemical penetration enhancers, membrane permeability agents, membrane transport agents, emollients, surfactants, stabilizers, and combination thereof. In some embodiments, the delivery vehicle composition can be administered as a liquid formulation, such as a solution or suspension, a semi-solid formulation, such as a lotion or ointment, or a solid formulation. In some embodiments, the delivery vehicle composition can be formulated as liquids, including solutions and suspensions, such as eye drops or as a semi-solid formulation, such as ointment or lotion for topical application to mucosa, such as the eye or vaginally or rectally. The formulation may contain one or more excipients, such as emollients, surfactants, emulsifiers, and penetration enhancers.

In some cases, a pharmaceutical composition may include a salt. A salt can be relatively non-toxic. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like.

In some cases, compositions provided herein can have a circulation half-life in a subject of about 6 hours, 12 hours, 18 hours, 24 hours, 30 hours, 36 hours, 42 hours, or 48 hours. In some embodiments compositions provided herein can comprise a circulation half-life of more than about 48 hours.

### Kits

Disclosed herein can be kits comprising agonist compositions. In some cases, a kit can include an agonist composition containing an effective amount of an agonist in unit dosage form. In some cases, a kit comprises a sterile container which can contain an agonist composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In some cases, a delivery vehicle composition can be dehydrated, stored and then reconstituted such that a substantial portion of an internal content is retained.

### EXAMPLES

### Example 1: BDNF to TrkB Signaling is Necessary for Activity-Regulated Oligodendrocyte Precursor Cell Proliferation

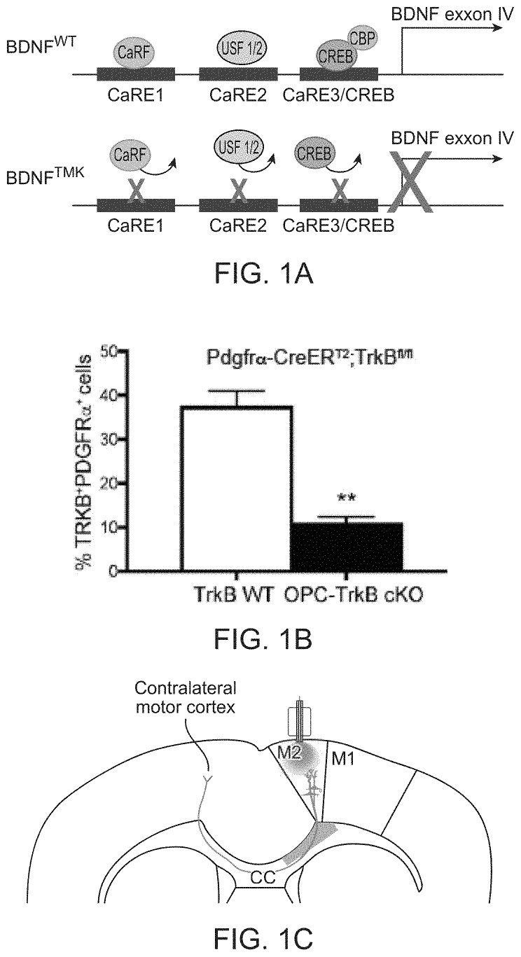

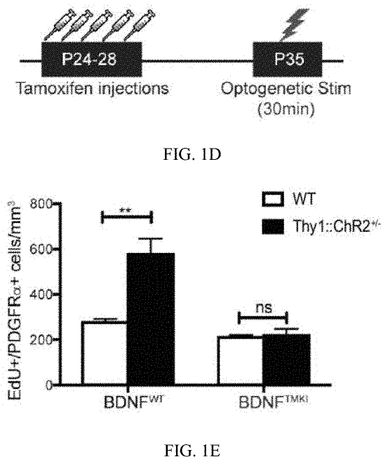

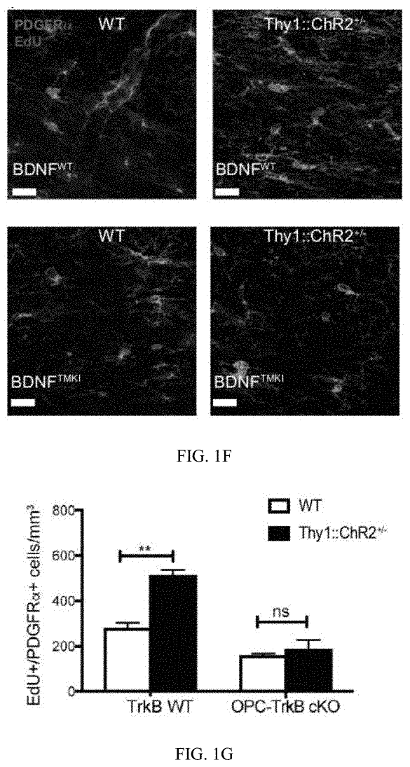

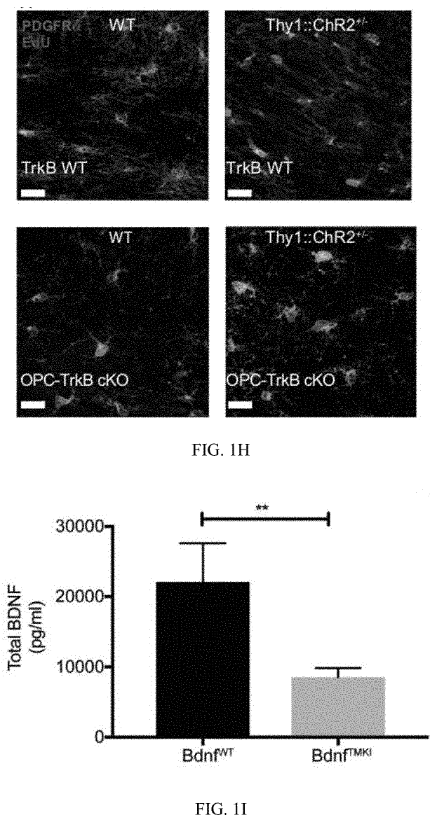

To test the role of BDNF to TrkB signaling in neuronal activity regulated oligodendrocyte precursor cell proliferation, we optogenetically stimulated neuronal activity of the premotor circuit in genetically engineered mouse models that either lack activity-regulated Bdnf expression, or the BDNF receptor TrkB in OPCs. In the first model, we used a mouse deficient in activity-induced expression of Bdnf due to knock in mutations at three calcium regulatory element binding sites in the Bdnf promoter IV: CaRE1, CaRE2 and CaRE3/CREB (BdnfTMKI, TMKI=triple-site mutant; FIG. 1A). These engineered mutations prevent the binding of calcium-regulated factors critical for neuronal-activity induced Bdnf transcription, particularly CREB, and specifically block activity-regulated Bdnf transcription while allowing activity-independent transcription to proceed. We find that in frontal cortex and subjacent corpus callosum tissue microdissected from unmanipulated mice in standard housing conditions, this loss of activity-regulated Bdnf expression results in a decrease in total BDNF protein levels by ˜50% (FIG. 1I). In the second model, Pdgfra-CreERT2 mice were bred to TrkBfl/fl mice to conditionally knockout the TrkB receptor specifically from OPCs. Using this conditional, inducible Pdgfra-CreERT2 driver mouse and a 5-day tamoxifen administration paradigm, we find recombination is achieved in ˜80% of OPCs. Induction of recombination with tamoxifen resulted in a robust decrease in OPC expression of TrkB, as measured by the percent of PDGFRa+ cells co-expressing TrkB (FIG. 1B).

To test the role of neuronal activity in activity-dependent oligodendrocyte proliferation, we performed optogenetic stimulation of premotor cortex projection neurons in awake, freely behaving mice. To accomplish this, we bred both the BdnfTMKI mouse and the Pdgfra-CreERT2; TrkBfl/fl (henceforth referred to as OPC-TrkB cKO) mouse to the well characterized Thy1::ChR2+/− mouse, in which expression of the excitatory opsin channelrhodopsin-2 is chiefly expressed in cortical layer V projection neurons. Placing an optical fiber in superficial premotor cortex allows for ˜10% of the light to penetrate to mid-cortex, stimulating the apical dendrites of ChR2-expressing layer V projection neurons. This optogenetic paradigm stimulates premotor circuit activity, evident by complex motor output in the form of unidirectional ambulation. Importantly, microglial inflammation occurs chiefly in the superficial cortex around the optical-neural interface, resolves within a week of optical fiber placement and does not involve the corpus callosum at any time point. We have previously shown that premotor cortex projection neuronal activity results in a circuit-specific increase in OPC proliferation and oligodendrogenesis in the premotor deep cortical layers and white matter projections in the corpus callosum, and we examined the same region of corpus callosum here (FIG. 1C).

Using these two genetic mouse models (BdnfTMKI; Thy1::ChR2+/− and OPC-TrkB cKO; Thy1::ChR2+/−), we sought to determine if loss of activity-dependent BDNF to TrkB signaling influences OPC proliferation in response to optogenetic stimulation of neuronal activity. The optical-neural interface was placed in the superficial premotor cortex (also known as M2 cortex) at postnatal day 28 (P28) and one week later (P35) mice underwent a single optogenetic stimulation (473 nm light, 20 Hz; 30s on/120s off over 30 min; FIG. 1D). Non-opsin expressing (“WT”) littermate control mice were identically manipulated to control for any effects of surgery, ferrule placement, or blue light exposure. The thymidine analogue EdU was administered at both the start and cessation of the 30-min stimulation session to mark cells proliferating during this time, and mice were sacrificed three hours after the end of the stimulation session. Optogenetic stimulation of the premotor cortex in BdnfWT; Thy1::ChR2+/− mice resulted in the expected increase in proliferating OPCs in the premotor projections within the corpus callosum, measured as the density of PDGFRa+ OPCs co-expressing EdU, compared to identically manipulated, non-opsin expressing (WT) littermate controls (FIG. 1E). In contrast, optogenetic stimulation of premotor circuit activity in mice that lack activity-regulated Bdnf expression (BdnfTMKI mice) exhibited no increase in OPC proliferation in response to neuronal activity (BdnfWT; Thy1::ChR2+/−: 582±63.68 cells/mm3 vs. BdnfWT; WT: 282±9.78 cells/mm3, p=0.0012; BdnfTMKI; Thy1::ChR2+/−: 224±24.34 cells/mm3 vs. BdnfTMKI; WT: 216±4.85 cells/mm3 p=0.9983; FIG. 1E, representative images FIG. 1F).

We next conditionally deleted the BDNF receptor specifically in OPCs in the OPC-TrkB cKO model. Tamoxifen was administered for five consecutive days (100 mg/kg, P24-28) to all mice in the four experimental groups, including OPC-TrkB cKO mice with and without ChR2 expression and TrkB WT (no Cre driver) littermates with and without ChR2 expression (OPC-TrkB cKO; Thy1::ChR2+/−, OPC-TrkB cKO; WT, TrkB WT; Thy1::ChR2+/−, TrkB WT; WT). Seven days after the final tamoxifen dose, mice in each of these four experimental groups received a single optogenetic stimulation session as above. As expected, premotor optogenetic stimulation resulted in an increase in OPC proliferation in TrkB WT; Thy1::ChR2+/− mice relative to identically manipulated, non-opsin expressing littermate control mice (TrkB WT; WT; FIG. 1G). However, optogenetic stimulation of mice with conditional knockout of TrkB expression in OPCs did not result in an increase in OPC proliferation (TrkB WT; Thy1::ChR2+/−: 515±20.12 cells/mm3 vs. TrkB WT; WT: 279.63±22.67 cells/mm3, p=0.001; OPC-TrkB cKO; Thy1::ChR2+/−: 186.42±41.24 cells/mm3 vs. OPC-TrkB cKO; WT: 158.71±7.56 cells/mm3, p=0.8774; FIG. 1G, representative images FIG. 1H). Thus, loss of activity-regulated Bdnf expression or loss of TrkB expression in OPCs completely abrogates activity-regulated OPC proliferation in the premotor circuit.

### Example 2: BDNF to TrkB Signaling is Necessary for Activity-Regulated Oligodendrogenesis

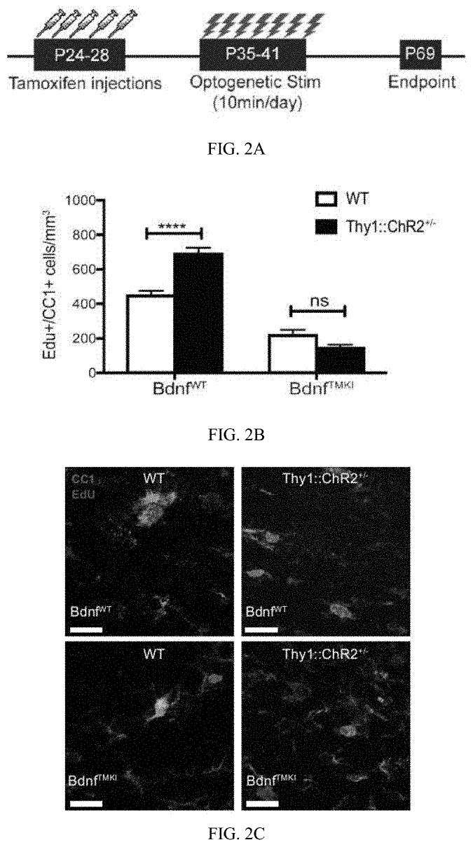

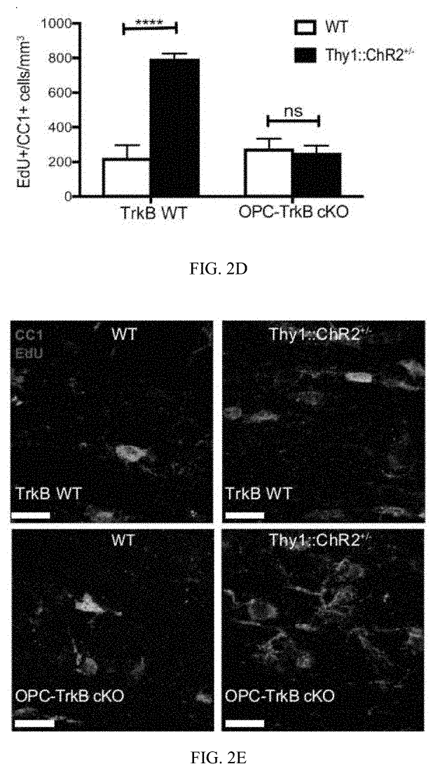

To test the role of activity-dependent BDNF to TrkB signaling in adaptive oligodendrogenesis, we evaluated newly generated oligodendrocytes one month following the end of a one-week paradigm of daily optogenetic stimulation. As above, the optical-neural interface was placed in premotor cortex at P28, and a week later (P35) mice received daily optogenetic stimulation for 7 consecutive days (473 nm, 20 Hz; 30s on/120s off over 10 min; FIG. 2A). EdU was administered at the start of each daily stimulation to fate map cells dividing at the time of stimulation. As expected, optogenetic stimulation of the premotor cortex in BdnfWT; Thy1::ChR2+/− mice resulted in an increase in newly generated mature oligodendrocytes in the premotor projections in the corpus callosum, identified as EdU-marked cells co-expressing CC1 at one month following the optogenetic stimulation paradigm (FIG. 2B). In contrast, BdnfTMKI mice that lack activity-regulated Bdnf expression exhibited no increase in EdU-marked, CC1 co-expressing oligodendrocytes within the premotor circuit (BdnfWT; Thy1::ChR2+/−: 695.30±31.11 cells/mm3 vs. BdnfWT; WT: 453.46±22.67 cells/mm3, p<0.0001; BdnfTMKI; Thy1::ChR2+/−:151.15±12.77 cells/mm3 vs. BdnfTMKI; WT: 224.21±25.10 cells/mm3, p=0.2324; FIG. 2B, representative images FIG. 2C). To determine whether TrkB signaling in OPCs is similarly necessary for activity-regulated oligodendrogenesis, premotor circuit optogenetic stimulation was performed in mice with OPC-specific, inducible deletion of TrkB (OPC-TrkB cKO) or with intact TrkB expression as described above. As expected, optogenetic stimulation of TrkB WT; Thy1::ChR2+/− mice resulted in an increase in newly generated, EdU-marked oligodendrocytes at one month following the end of the week-long optogenetic stimulation paradigm (FIG. 2D), and this activity-regulated increase in oligodendrogenesis was completely abrogated in mice lacking expression of TrkB in OPCs (TrkB WT; Thy1::ChR2+/−: 795.30±31.11 cells/mm3 vs. TrkB WT; WT: 222.95±73.11 cells/mm3, p<0.0001; OPC-TrkB cKO; Thy1::ChR2+/−: 251.15±42.77 cells/mm3 vs. OPC-TrkB cKO; WT: 275.85±58.99 cells/mm3, p=0.9872; FIG. 2D, representative images FIG. 2E). Taken together, the data indicate that BDNF to TrkB signaling is required for activity-regulated oligodendrogenesis in the premotor circuit.

### Example 3: BDNF to TrkB Signaling is Necessary for Activity-Regulated Myelination

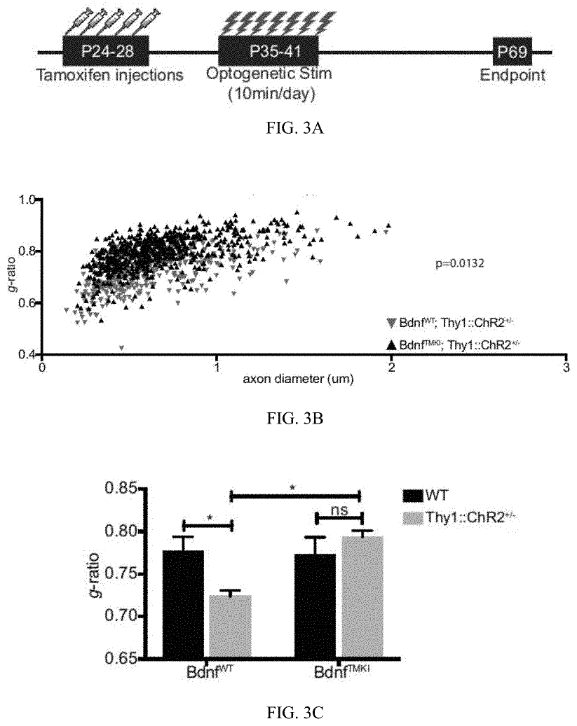

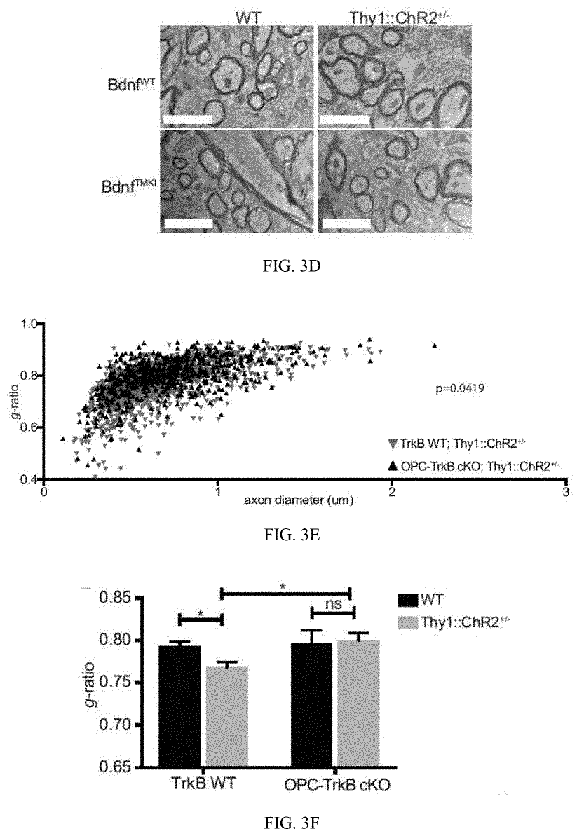

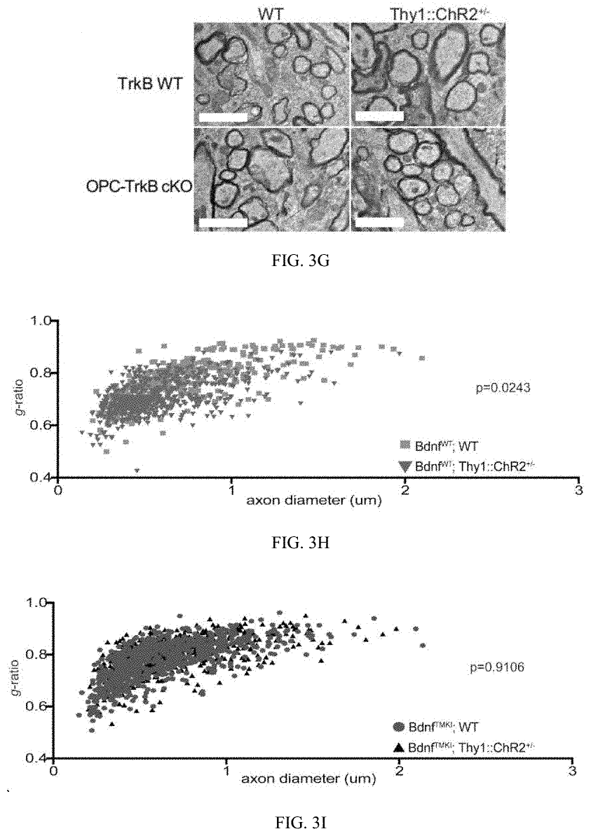

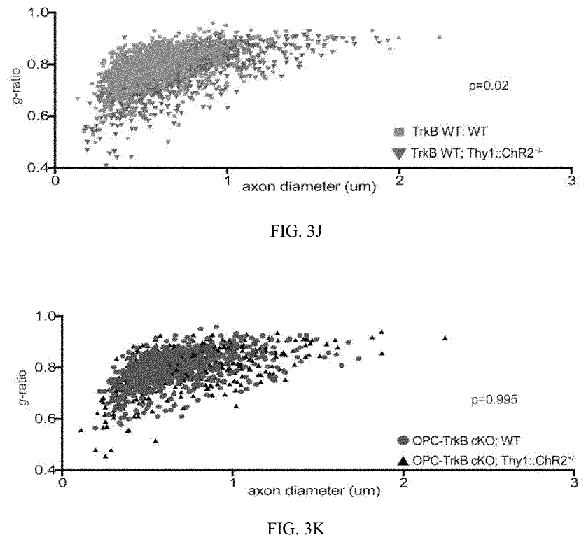

We next sought to determine the influence of BDNF to TrkB signaling in activity-regulated myelin microstructure changes in these mouse models. As above, the week-long optogenetic stimulation paradigm was administered to P35 mice (FIG. 3A). Four weeks after the completion of optogenetic stimulation, mice were sacrificed and prepared for transmission electron microscopy (EM) analysis, measuring myelin sheath thickness relative to axon caliber (g-ratio, the diameter of the axon divided by the diameter of the axon plus myelin sheath) in the premotor projection fibers at the level of the cingulum. Optogenetically stimulated BdnfWT; Thy1::ChR2+/− mice exhibited thicker myelin (indicated by lower g-ratios), consistent with our previous observations. In contrast, optogenetically stimulated BdnfTMKI; Thy1::ChR2+/− mice exhibited no change in myelin thickness as compared to either identically manipulated BDNFTMKI; WT (no opsin) and BDNFWT; WT (no opsin) mice (BdnfWT; Thy1::ChR2+/−: g-ratio 0.724±0.007 vs. BdnfWT; WT: g-ratio 0.777±0.017, p=0.0243; BdnfTMKI; Thy1:: ChR2+/−: g-ratio 0.794±0.007 vs. BdnfTMKI; WT: g-ratio 0.773±0.020, p=0.3921; FIG. 3B-FIG. 3C, FIG. 3H-FIG. 3I; representative images FIG. 3D). Similarly, when we repeat this experiment in the OPC-TrkB cKO model, we find that TrkB WT; Thy1::ChR2+/− mice exhibit the expected increase in myelin thickness following optogenetic stimulation of premotor neuronal activity, and this myelin change was completely abrogated in mice lacking TrkB expression in OPCs (TrkB WT; Thy1::ChR2+/−: g-ratio 0.768±0.006 vs. TrkB WT; WT: g-ratio 0.793±0.006, p=0.020; OPC-TrkB cKO; Thy1::ChR2+/−: g-ratio 0.799±0.009 vs. OPC-TrkB cKO; WT: g-ratio 0.796±0.016; p=0.995; FIG. 3E-FIG. 3F, FIG. S2C-D, representative images FIG. 3G).

### Example 4: Failure of Adaptive Myelination in a Mouse Model of Chemotherapy-Induced Neurocognitive Impairment

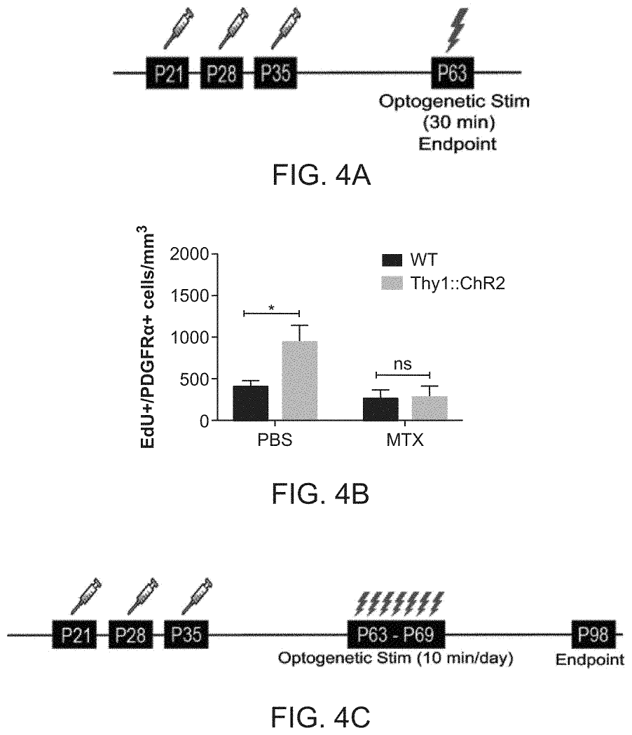

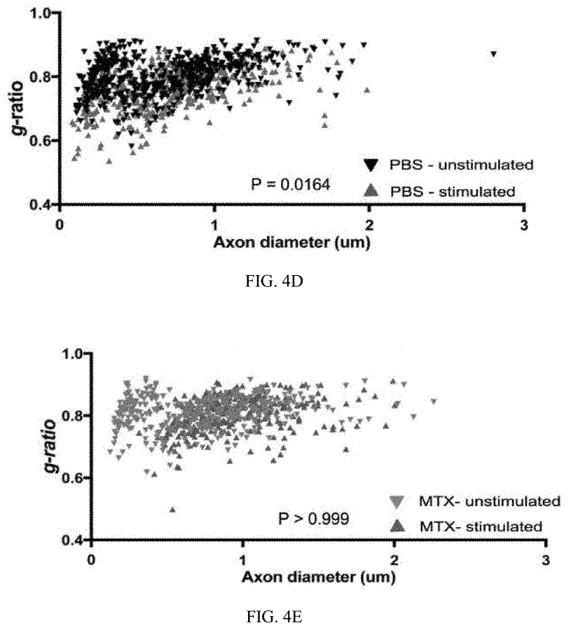

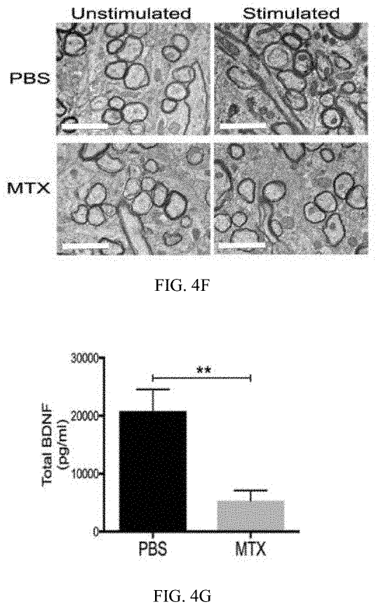

Chemotherapy exposure frequently results in a lasting and debilitating neurological syndrome characterized by impaired attention, concentration, multitasking, memory and motor functions. We have recently developed a mouse model of juvenile chemotherapy exposure to the anti-metabolite chemotherapeutic agent methotrexate (MTX), a commonly used agent for many forms of cancer and is particularly associated with long-term neurological dysfunction. As described in the accompanying manuscript, MTX chemotherapy administered to mice at doses that achieve clinically-relevant serum and brain drug concentrations results in persistent dysregulation of white matter OPC population dynamics. Mice exposed to MTX at P21, 28, and 35 exhibit a lasting reduction of OPCs in the corpus callosum at one month (P63) and 6 months (P203) following cessation of MTX exposure together with complex alterations of the gliogenic microenvironment (see accompanying manuscript). To ascertain if MTX chemotherapy exposure in this model also disrupts neuronal activity-regulated myelination, we tested the influence of optogenetically stimulated premotor circuit activity, as above, in mice with previous MTX chemotherapy exposure. Thy1::ChR2+/− and WT (no opsin) mice were exposed to MTX (100 mg/kg i.p.) or PBS vehicle on P21, 28 and 35. The optical-neural interface was placed at P56 in M2 superficial cortex as described above. Mice underwent a single optogenetic stimulation session at P63 (473 nm light, 20 Hz; 30s on/120s off over 30 min), received EdU before and after the session, and were sacrificed 3-hours later as above (FIG. 4A). Photogenetically stimulated PBS vehicle control-treated mice exhibited the expected increase in EdU-marked, proliferating OPCs compared to identically manipulated WT (no opsin) controls (PBS vehicle-treated, Thy1::ChR2+/: 952±188 EdU+/PDGFRa+ cells/mm3 vs. PBS vehicle, WT: 415±61 EdU+/PDGFRa+ cells/mm3, p=0.0218; FIG. 4B). In contrast, Thy1::ChR2+ mice exposed to remote MTX chemotherapy did not exhibit activity-dependent OPC proliferation in comparison to identically manipulated WT (no opsin) controls (MTX-treated, Thy1::ChR2+/−: 293±120 EdU+/PDGFRa+ cells/mm3 vs. MTX-treated, WT: 269±101 EdU+/PDGFRa+ cells/mm3, p>0.999; FIG. 4B). These data demonstrate a failure of activity-dependent oligodendroglial lineage cell response following chemotherapy exposure.

To determine if activity-regulated myelin changes accompany the failure of activity-regulated OPC proliferation following MTX chemotherapy, we exposed Thy1::ChR2+/− mice to MTX or PBS vehicle control at P21, 28 and 35 followed by optical-neural interface placement at P56. Mice were optogenetically stimulated or identically manipulated with the exception of blue light exposure (surgically manipulated, fiber optic connected but no blue light exposure) using the one-week paradigm described above from P63-69 (473 nm, 20 Hz; 30s on/120s off over 10 min). Four weeks following the end of the stimulation paradigm (P98), myelin sheath thickness was assessed using EM at the level of the cingulum of the corpus callosum (FIG. 4C). As expected, optogenetically stimulated Thy1::ChR2+/− mice exposed to PBS exhibited an increase in myelin sheath thickness compared to unstimulated PBS-exposed mice (g-ratio: 0.79±0.009 unstimulated vs. 0.75±0.014 stimulated, p=0.0164; FIG. 4D and FIG. 4F). This neuronal activity-dependent increase in myelin thickness was completely abrogated in mice exposed to previous methotrexate chemotherapy (g-ratio: 0.81±0.008 unstimulated vs 0.81±0.01 stimulated, p>0.999; FIG. 4E-FIG. 4F). Taken together, these data demonstrate a failure of adaptive myelination following methotrexate chemotherapy in this model of chemotherapy-related cognitive impairment.

### Example 5: Reduced White Matter BDNF Levels Following Chemotherapy Exposure

Given the lasting microenvironmental dysregulation that follows MTX chemotherapy exposure and the role for BDNF signaling in adaptive myelination demonstrated above, we asked whether decreased BDNF protein levels accompanied this failure of adaptive myelination after methotrexate chemotherapy exposure. Mice were exposed to the methotrexate (MTX) paradigm (doses on P21, P28 and P35) and then sacrificed 4 weeks after the final MTX dose, on P63. The corpus callosum was microdissected and BDNF protein levels were assayed by ELISA. Mice with previous MTX exposure exhibited a more than three-fold decrease in corpus callosum BDNF protein levels compared to PBS vehicle controls (MTX: 5347±1736 μg/ml vs. PBS: 20818±3717 μg/ml, p=0.003, FIG. 4G).

### Example 6: A Small Molecule TrkB Partial Agonist Recapitulates the Effects of BDNF on Oligodendrogenesis

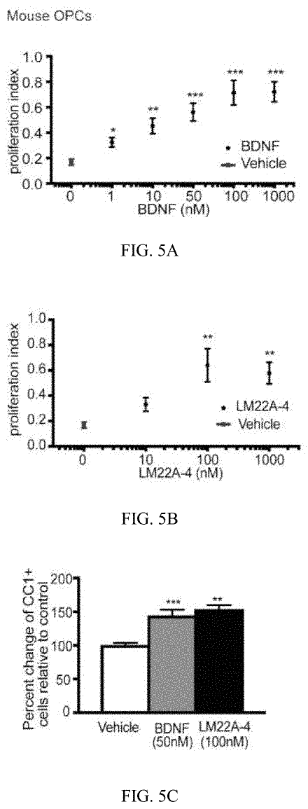

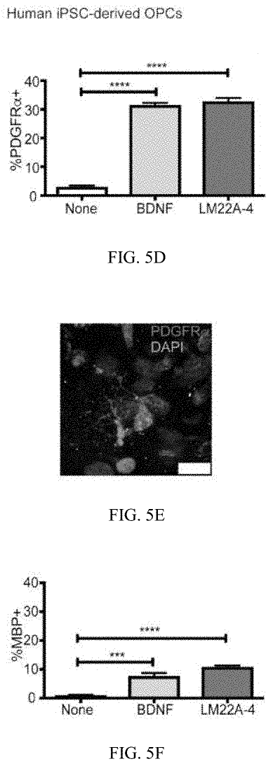

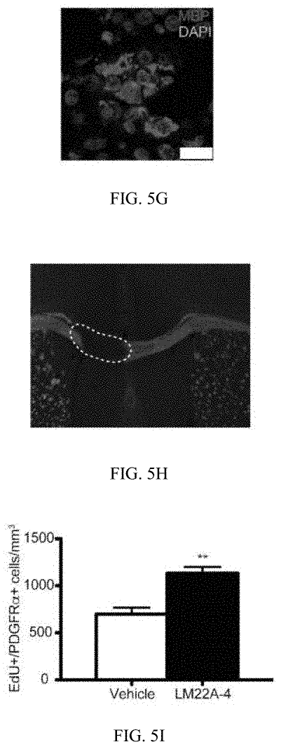

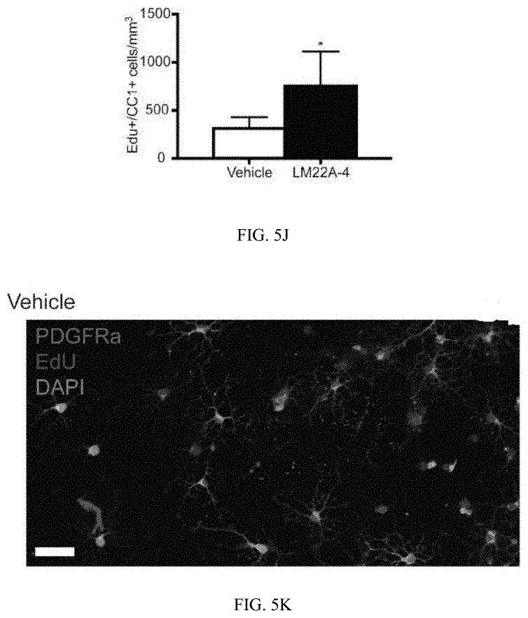

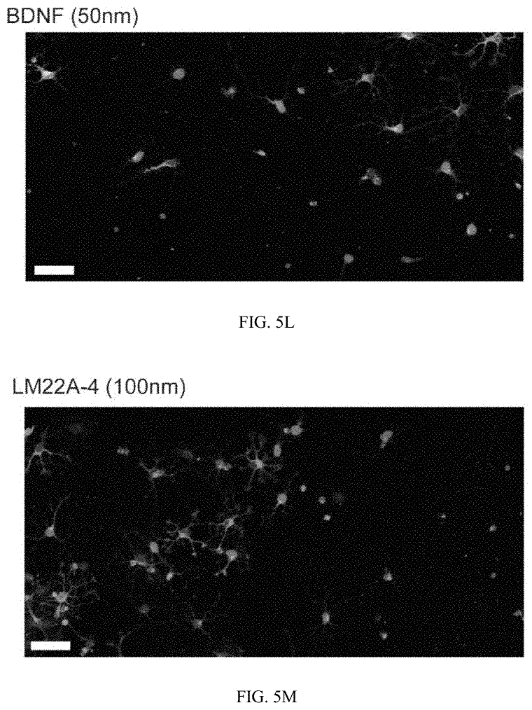

While BDNF signaling is an attractive therapeutic strategy for many diseases, the short plasma half-life and poor blood-brain-barrier penetration of BDNF protein limits therapeutic possibilities of recombinant protein administration. Thus, to therapeutically manipulate BDNF-TrkB signaling to promote oligodendrogenesis, we tested a small molecule TrkB partial agonist, LM22A-4. This compound was previously shown to activate the TrkB receptor specifically and to promote TrkB activation and neuronal survival in vitro and in vivo following systemic administration in multiple mouse models of neurological diseases. To determine if LM22A-4 could promote OPC proliferation and differentiation in a manner similar to BDNF, we compared the effects of recombinant BDNF, LM22A-4 or vehicle control in cultures of murine OPCs. Following a 24-hour exposure, we found that BDNF treatment increased the proliferative index (fraction of total cells in S phase, measured by co-localization of PDGFRa with EdU) of OPCs in vitro in a dose dependent fashion (FIG. 5A, representative image FIG. 5K and FIG. 5L), as expected. LM22A-4 similarly increased the proliferative index of OPCs in the low nanomolar range and in a dose-dependent manner (FIG. 5B, representative image FIG. 5K and FIG. 5M). Following a seven-day exposure, BDNF and LM22A-4 similarly promoted oligodendroglial differentiation, as measured by an increase in cells expressing the mature oligodendrocyte marker CC1 relative to vehicle control (FIG. 5C). We next tested the effects of BDNF and LM22A-4 in human iPSC-derived OPCs, and found that BDNF and LM22A-4 similarly promote in vitro oligodendrogenesis in human oligodendroglial lineage cells (FIG. 5D-FIG. 5G).

To investigate the effects of LM22A-4 treatment on OPC proliferation and oligodendrogenesis in vivo, we next examined mice treated with either LM22A-4 or vehicle control in a well-established demyelination model. Stereotaxic injection of lysolecithin into white matter regions causes a focal, toxic, demyelinating injury; this leads to microglial activation and demyelination, followed by OPC proliferation, recruitment and migration into the lesion area. Mice received a stereotactic injection of lysolecithin in the corpus callosum and were then treated beginning on post-injury day 7 with LM22A-4 (50 mg/kg i.p.) or vehicle control for one week (ending on 14 days after lysolecithin injection, representative image of lesion FIG. 5H). EdU was also administered systemically during the treatment period to mark proliferating cells. Mice that received LM22A-4 treatment exhibited increased OPC proliferation specifically within the lesioned area, as measured by co-localization of EdU with PDGFRa+ OPCs (699.6±68.97 EdU+/PDGFRa+ cells/mm3 in vehicle-treated mice vs. 1132±67.1 EdU+/PDGFRa+ cells/mm3 in LM22A-4 treated mice, p=0.002, FIG. 5I). Next, we repeated this experimental paradigm but with administration of LM22A-4 for a longer duration (from day 7 to 21 following lysolecithin injury) to examine changes in oligodendrocyte differentiation. Again, EdU was injected for the first 7 days of treatment to fate map dividing cells. LM22A-4 treatment increased newly generated mature oligodendrocytes in the lesioned area of the corpus callosum, identified by EdU-marked cells co-expressing the oligodendrocyte marker CC1 (326.1±46.55 EdU+/CC1+ cells/mm3 in vehicle treated mice vs. 764.8±141.6 EdU+/CC1+ cells/mm3 in LM22A-4 treated mice, p=0.0241, FIG. 5J). Thus, LM22A-4 recapitulates the expected influence of BDNF to TrkB signaling on oligodendroglial lineage precursor proliferation and differentiation at baseline and in the context of re-myelination.

### Example 7: TrkB Partial Agonist LM22A-4 Normalizes Myelination and Rescues Behavioral Function after Methotrexate Chemotherapy Exposure

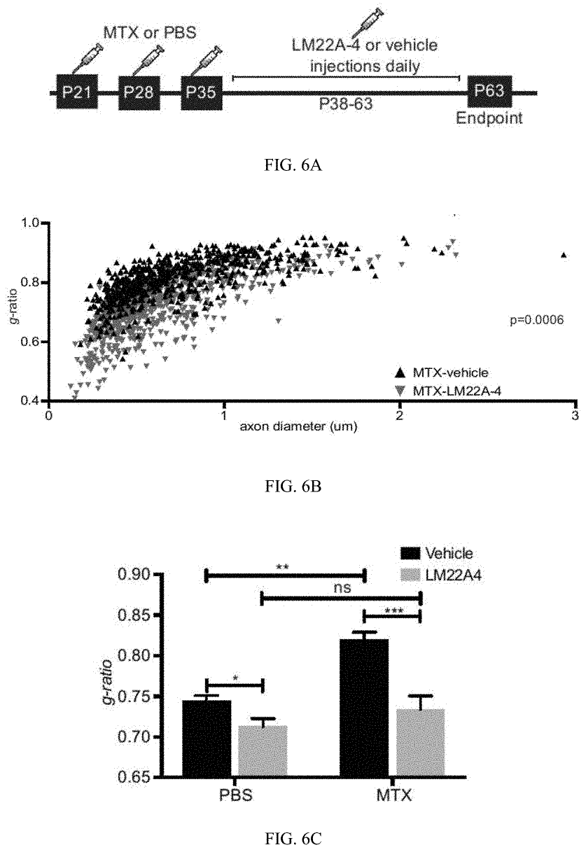

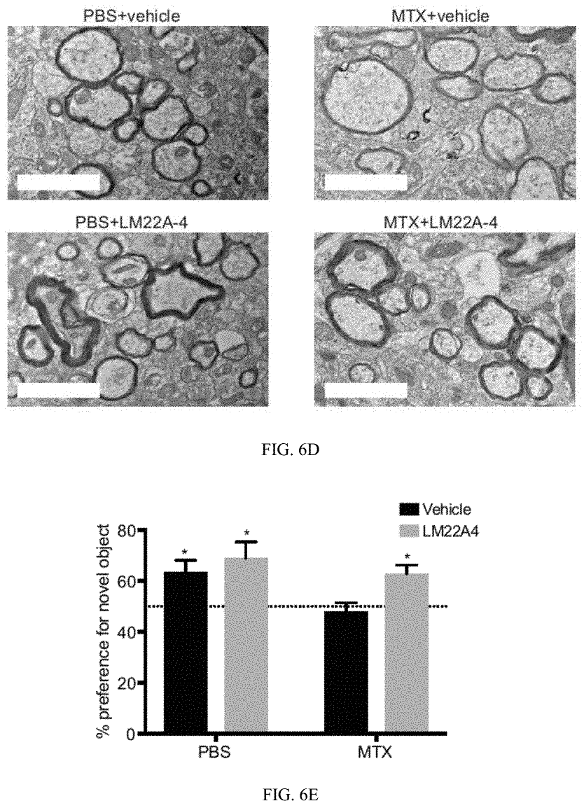

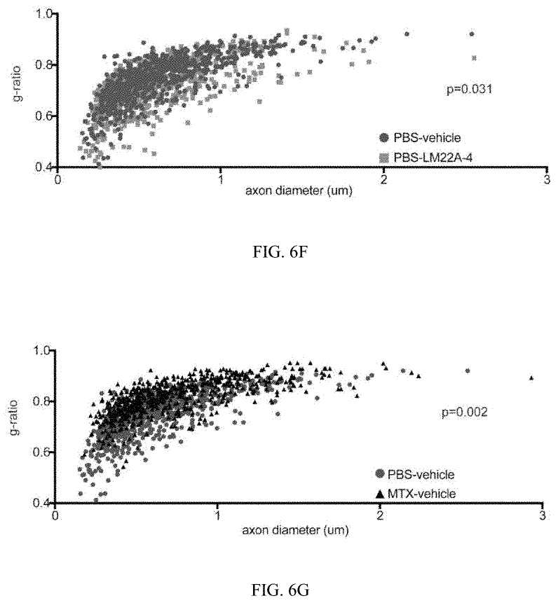

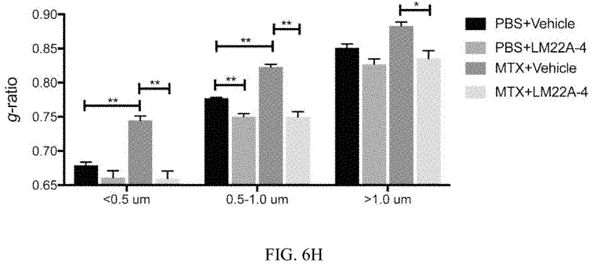

The chemotherapy-induced neurological syndrome is characterized by decreased attention and memory function and this is recapitulated in the MTX mouse model used here. To test the hypothesis that BDNF to TrkB signaling could promote myelination and improve cognitive dysfunction after chemotherapy exposure, mice were subjected to the above MTX chemotherapy protocol, then treated systemically with the TrkB partial agonist LM22A-4 (50 mg/kg i.p. daily, P38-P62, FIG. 6A) or vehicle control, and analyzed at 24 hours after the final injection (P63) for changes in myelin microstructure and behavioral performance in the novel object recognition test.

Myelin microstructure in the cingulum of the corpus callosum was examined by transmission electron microscopy. Mice who received MTX chemotherapy and vehicle control rather than LM22A-4 exhibited decreased myelin thickness, as indicated by increased g-ratios of myelinated axons in the cingulum, consistent with the results reported in the accompanying manuscript. In contrast, MTX mice that were treated with LM22A-4 exhibited a significant increase in myelin thickness as compared with the vehicle control-treated group (MTX+vehicle: g-ratio 0.820±0.00; PBS+vehicle: g-ratio 0.745±0.006; MTX+LM22A-4: g-ratio 0.733±0.017; PBS+LM22A-4: g-ratio 0.713±0.010, MTX+vehicle vs. PBS+vehicle p=0.002, MTX+LM22A-4 vs. MTX+vehicle p=0.0006; FIG. 6B-FIG. 6C, FIG. 6F, and FIG. 6G, representative images 6D). LM22A-4 treatment of MTX-treated mice increased myelin thickness in small, medium and large caliber axons (FIG. 6H). The myelin microstructural effects of LM22A-4 thus mimic activity-regulated changes in myelin microstructure, but would lack the circuit-specificity of the activity-regulated response. To test the influence of LM22A-4 on a behavioral measure of attention and memory function, the novel object recognition test was used. After brief introduction to two identical objects, mice are then exposed to one familiar object as well as a completely novel object after a time delay of 5-minutes. Healthy mice spend significantly more time exploring the new object, while mice experiencing deficits in attention or memory are unable to recognize either object as new, and therefore spend equal time with each object. As expected, PBS vehicle control-treated mice interact with the novel object significantly more than chance. In contrast, MTX-treated mice demonstrate no preference for either object as indicated by time spent with the novel and the familiar object (FIG. 6E), consistent with the results reported in the accompanying manuscript. Treatment with LM22A-4 from P38-P62 rescues performance in the novel object recognition test in MTX treated mice as these mice spend a significant percentage of their time interacting with the novel object over the familiar object (MTX+vehicle: 47.94%±3.50%, p=0.5968 over 50%; PBS+vehicle: 63.37%±4.72%, p=0.0219 over 50%; MTX+LM22A-4: 62.81%±3.31%, p=0.0305 over 50%; PBS+LM22A-4: 68.93%±6.34%, p=0.0317 over 50%; FIG. 6E). Taken together, these data demonstrate that LM22A-4 normalizes myelin sheath thickness and rescues behavioral deficits in attention/memory function following chemotherapy exposure, suggesting the therapeutic approach of promoting TrkB receptor activation for people suffering from the chemotherapy-related cognitive impairment.

1-45. (canceled)

46. A method of preventing or treating a cancer therapeutic-related cognitive dysfunction, comprising administering to a subject in need thereof an effective amount of a TrkB agonist, wherein said TrkB agonist is administered to the subject before, during, or after completion of the administration of a cancer treatment regimen or a combination thereof.

47. The method of claim 46, wherein the cancer treatment regimen comprises one or more doses of chemotherapy, one or more doses of radiation therapy, or one or more doses of immunomodulatory therapy.

48. The method of claim 46, wherein the method further comprises administering to the subject a test of cognitive function prior to administration of the TrkB agonist.

49. The method of claim 48, wherein the test of cognitive function indicates that the subject has a cognitive dysfunction.

50. The method of claim 48, wherein the test of cognitive function is selected from the General Practitioner Assessment of Cognition (GPCOG), Memory Impairment Screen (MIS), MiniMental State Exam (MMSE), and the Six Item Cognitive Impairment Test (6CIT).

51. The method of claim 46, wherein the cognitive dysfunction comprises one or more symptoms selected from: being unusually disorganized, confusion, difficulty concentrating, difficulty finding the right word, difficulty learning new skills, difficulty multitasking, fatigue, feeling of mental fogginess, short attention span, short-term memory problems, taking longer than usual to complete routine tasks, trouble with verbal memory, adynamia, lack of motivation, impulsiveness, disinhibition, verbosity, tangentiality, irritability, fidgety, low frustration tolerance and trouble with visual memory.

52. The method of claim 46, wherein the method further comprises administering a test of cognitive function after administration of an effective amount of the TrkB agonist.

53. The method of claim 52, wherein the test of cognitive function after administration of an effective amount of the TrkB agonist indicates maintenance or improvement in cognitive function of the subject, or improvement in one or more symptoms of cognitive dysfunction of the subject.

54. The method of claim 46, wherein said method comprises administering said TrkB agonist to the subject after completion of the administration of the cancer treatment regimen.

55. The method of claim 54, wherein said method comprises administering said TrkB agonist to the subject for about 1 month to about 2 years after completion of the administration of the cancer treatment regimen.

56. The method of claim 46, wherein said method comprises administering said TrkB agonist to the subject before the administration of the cancer treatment regimen.

57. The method of claim 46, wherein the method comprises administering said TrkB agonist to the subject during the cancer treatment regimen.

58. The method of claim 57, wherein the TrkB agonist is administered to the subject within six hours of administration of a dose of the cancer treatment regimen.

59. The method of claim 46, wherein the TrkB agonist is administered daily, every other day, every third day, or every fourth day.

60. The method of claim 46, wherein the method comprises promoting or maintaining growth of glial cells.

61. The method of claim 46, wherein the subject has a cancer selected from breast cancer, ovarian cancer, prostate cancer, leukemia, lymphoma, brain tumor, and sarcoma.

62. The method of claim 46, wherein the subject has a cancer selected from a cancer of the central nervous system.

63. The method of claim 46, wherein the TrkB agonist is selected from: N-Acetylserotonin, Amitriptyline, BNN-20, BNN-27, Brain-derived neurotrophic factor, Deoxygedunin, 7,8-Dihydroxyflavone, 4′-Dimethylamino-7,8-dihydroxyflavone, Diosmetin, HIOC, LM22A-4, LM22B-10, Neurotrophin-3, Neurotrophin-4, Norwogonin, R7, R13, and 7,8,3′-Trihydroxyflavone.

64. The method of claim 46, wherein the TrkB agonist is a small molecule mimetic of a brain-derived neurotrophic factor (BDNF) β-turn loop, wherein the β-turn loop is loop 2, and the small molecule mimetic has a structure of Formula (II): 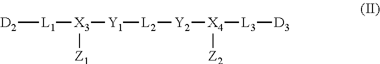
or a pharmaceutically acceptable salt thereof, wherein:
L₁ and L₃ are independently selected from the group consisting of C₁-C₅ alkylene, arylene, aralkylene, and substituted arylene;
L₂ is selected from the group consisting of C₁-C₅ alkylene, arylene, aralkylene, substituted arylene, 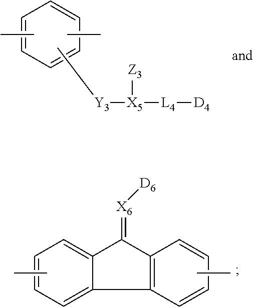
L₄ is C₁-C₅ alkylene;
Z₁, Z₂, and Z₃ are independently selected from the group consisting of H, alkyl, aryl, and aralkyl;
X₃, X₄, X₅, and X₆ are independently N or CH;
Y₁, Y₂, and Y₃ are independently carbonyl, sulfonyl, or methylene; and
D₂, D₃, D₄, and D₅ are independently selected from H, alkyl, halo, hydroxyl, mercapto, mercaptoalkyl, alkoxyl, aryloxyl, aralkoxyl, acyloxyl, carboxyl, alkyloxycarbonyl, aryloxycarbonyl, aralkoxycarbonyl, acylamino, carbamoyl, alkylcarbamoyl, dialkylcarbamoyl, 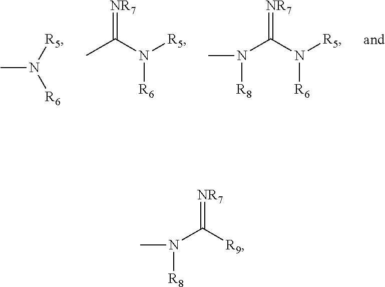
wherein R₅, R₆, R₇, R₈, and R₉ are independently selected from H, alkyl, aralkyl, and aryl.

65. The method of claim 64, wherein the compound is selected from the group consisting of: 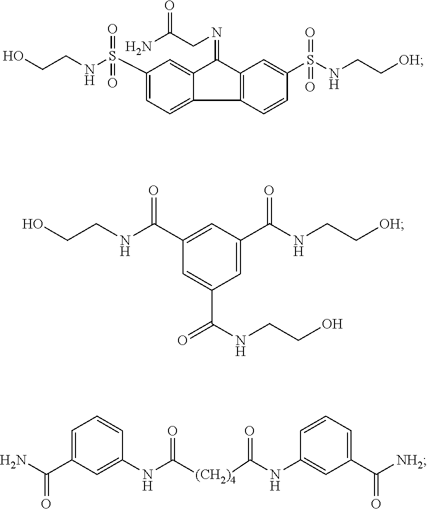
and a pharmaceutically acceptable salt of any one thereof.
